(19) United States
(12) Patent Application Publication
(10) Pub. No.: US 2008/0096894 A1
(43) Pub. Date: Apr. 24, 2008

(54) Anti-Bacterial Compounds

(75) Inventors: Katy Jane Parker, Birmingham (GB); Daniel Lee Rathbone, Birmingham (GB); Peter Anthony Lambert, Birmingham (GB); Michael Damian Coleman, Birmingham (GB); Anthony Craig Hilton, Birmingham (GB); Antony Worthington, Birmingham (GB); David Charles Billington, Cedex (FR)

Correspondence Address: MICHAEL BEST & FRIEDRICH LLP, ONE SOUTH PINCKNEY STREET, P O BOX 1806, MADISON, WI, 53701, US

(73) Assignee: ASTON UNIVERSITY, Birmingham (GB)

(21) Appl. No.: 11/813,828

(22) PCT Filed: Jan. 12, 2006
(86) PCT No.: PCT/GB06/00101

§ 371 Date: Jul. 12, 2007

(30) Foreign Application Priority Data
Jan. 12, 2005 (GB) 0500568.1
Jul. 25, 2005 (GB) 0515249.1

(51) Int. Cl. A61K 31/497; A61K 31/47; A61K 31/44; A01N 43/42; A01N 43/60; A01P 1/00; A61P 31/04; C07D 241/02; C07D 215/48; C07D 213/86

(52) U.S. Cl. 514/252.1; 514/311; 514/357; 546/176; 546/329; 546/332; 544/336

(57) ABSTRACT

Compounds of formula (I), or salts or solvates thereof, in vitro as inhibitors of growth of Gram-positive bacteria, where A is selected from formula (a).

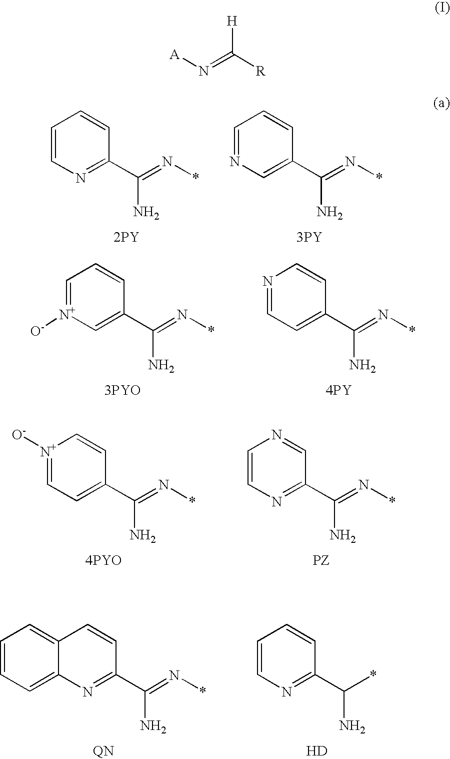

The present invention resides in the use of compounds as inhibitors of growth of Gram-positive bacteria, and some of the compounds themselves. The inhibitory characteristics of these compounds may find application in culture medium or as a treatment for bacterial infection.

The global rise of bacteria and other microorganisms resistant to antibiotics and antimicrobials in general, poses a major threat. Deployment of massive quantities of antimicrobial agents into the human ecosphere during the past 60 years has introduced a powerful selective pressure for the emergence and spread of antimicrobial-resistant bacterial pathogens. Resistant organisms of special epidemiological importance, due to the preponderance of these pathogens to cause cross-infection in hospitals and other health care settings, include methicillin-resistant Staphylococcus aureus (MRSA) and other Gram-positive bacteria such as vancomycin-resistant enterococci (VRE) and Clostridium difficile, and Streptococcus pneumoniae which is becoming increasingly resistant to P-lactams and other antimicrobials. Staphylococcus aureus S. aureus is an important cause of community- and hospital-acquired infection and is the second most important cause of septicaemia after Escherichia coli and the second commonest cause of line-associated infection and continuous ambulatory peritoneal dialysis peritonitis. S. aureus is also a major cause of bone, joint and skin infection. Overall, S. aureus is the commonest bacterial pathogen in modern hospitals and communities. It is also one of the most antimicrobial resistant and readily transmissible pathogens which, on average, may be carried by about a third of the normal human population, thus facilitating world-wide spread of epidemic strains.

By the early 1950s, resistance to penicillin, conferred by a penicillinase (=β-lactamase) born on transmissible plasmids, was common in strains of S. aureus acquired in hospitals. Alternative antimicrobial agents, namely tetracycline, streptomycin and the macrolides, were introduced, but resistance developed rapidly. The understanding of the chemistry of the P-lactam ring enabled the development of methicillin, a semisynthetic penicillinase-stable isoxazolyl penicillin. Methicillin and the subsequent development of other isoxazolyl semisynthetic agents such as flucloxacillin, cloxacillin and oxacillin, revolutionised the treatment of S. aureus infections.

MRSA were first detected in England in 1960 and have since become a well recognised cause of hospital-acquired infection world-wide. MRSA are resistant to all clinically available p-lactams and cephalosporins and readily acquire resistant determinants to other antimicrobial agents used in hospital medicine. Selective pressure has ensured the rise and world-wide spread of MRSA.

C. difficile

C. difficile is documented as the second major cause of hospital acquired infection next to methicillin resistant Staphylococcus aureus (MRSA) and is associated with the overuse of antibiotics, in particular, the cephalosporins (Barbut, F & Petit, J. C., CMI 2001, 405-410). However, in many of our hospitals, infection rates are substantially higher than the infamous MRSA. The clinical spectrum of disease associated with C. difficile ranges from antibiotic-associated diarrhea to potentially fatal pseudomembranous colitis (Barbut & Petit, supra).

C. difficile is a Gram-positive spore producing anaerobic bacterium, the spores of which contaminate the environment facilitating rapid spread of infection within the hospital environment. Cases of infection associated with C. difficile have increased over the past decade. In 1990, the number of reported cases within the UK was less than 1,000 compared to 43,672 in 2004. Furthermore, latest figures show there were 934 deaths due to C. difficile in 2003 which is a 38% rise compared to 2001 (HPA. Voluntary reporting of Clostridium difficile, England, Wales and Northern Ireland 2004., Commun Dis Rep Wkly, 19, 1-3 (2005)).

C. difficile establishes itself within the hospital environment and causes infection as it produces hardy spores which resistant to common methods of cleaning and disinfection which can persist on skin, clothes, bedding and furniture thus transmitting the infection to new patients. Only a few liquid chemical disinfectants possess sporicidal activity (iodine, chorine, aldehyde and peroxygen), therefore new antimicrobial compounds with potent Gram positive antimicrobial activity which are also sporicidal are needed.

Vancomycin-Resistance

S. aureus/MRSA

A further development is the ability of some strains to acquire increased or intermediate resistance to glycopeptides. Glycopeptide antibiotics, vancomycin in particular, have been the drugs of choice, and in many cases the only active agents, for treating infection with MRSA and other resistant Gram-positive bacteria such as enterococci. If MRSA are not controlled, then the clinical use of vancomycin or teicoplanin rises because of the increased number of wound and blood stream infections in hospitalised patients.

Enterococci

Enterococci, particularly Enterococcus faecium and E. faecalis, are primarily gut commensals but can become opportunistic pathogens that colonise and infect immunocompromised hosts, such as liver transplant patients. Vancomycin-resistant E. faecium (VREF) emerged and have since become important nosocomial pathogens. E. faecium resistant to gentamicin, vancomycin and other agents, have caused infections for which no therapeutic agents had been available in the UK, although quinupristin/dalfopristin, which is active (MIC ≦2 mg/L) against 86% of E. faecium isolates, has now been licensed. In the USA, the proportion of VREF among enterococci isolated from blood cultures increased from 0% in 1989 to 25.9% in 1999.

Thus, there is clearly a need for the development of new antimicrobial agents, particularly ones which are active against drug-resistant strains.

In the microbiology laboratory, bacteria are routinely cultured on nutrient media that may be in liquid (broth) form or solidified with the addition of agar in Petri dishes. Media are purchased from major suppliers as a freeze-dried powder that is reconstituted with water and sterilized by autoclaving. It is not uncommon to prepare selective bacteriological agars which contain supplements to allow the isolation of specific strains from mixed bacterial populations. Often these supplements are combinations of antibiotics or other inhibitory agents which are prepared separately and added to the cooling agar base. One limitation of these supplements in that they usually require refrigerated storage (which limits their distribution in developing countries). In addition, the supplements are often antibiotics (such as vancomycin) and their unregulated inclusion in culture media may contribute further to the development of bacterial resistance referred to above (this is particularly worrying with regard to vancomycin the current antibiotic of last resort) and their separate addition to pre-sterilised culture media provides the opportunity for contamination.

If an alternative can be found, Vancomycin would be less generally used. This is important since the use of vancomycin needs to be conserved in order to slow down the inevitable rise of vancomycin-resistant organisms. Vancomycin is also relatively expensive. Any alternative should preferably be cheaper to produce compared to vancomycin.

Other Hospital Acquired Infections

Hospital acquired infections (HAI) including those associated with prosthetic devices, surgical wounds, intravascular access and the urinary tract are a major cause of morbidity and mortality (National Audit Office. The Challenge of Hospital Acquired Infection. London: Stationary Office 2001). In the UK, It is estimated that nine percent of in-patients have a HAI at any one time which equates to approximately 300,000 infections per year. The outcome of HAI may range from discomfort to prolonged or permanent disability and as many as 5,000 patients per year die as a result. Furthermore, NHS costs associated with HAI are as much as £1 billion pounds per annum.

HAI arising from intravascular catheterisation and post-operative infection, e.g. septic loosening of prosthetic joints, results in significant morbidity and mortality. Indeed, catheter related infection is the major cause of sepsis in the western world whilst an infection rate of 1% is associated in total hip arthroplasty (Garvin, K. L & Hanssen, A. D., Journal of Bone and Joint Surgery [Am], 77-A, 1576-1588 (1995)). The majority of infections associated with surgery are due to Gram positive skin microorganisms including coagulase negative staphylococci in particular Staphylococcus epidermidis and also the anaerobic coryneform Propionibacterium acnes (also associated with the skin condition, acne). Microorganisms such as S. epidermidis and P. acnes reside on the surface of the skin and also in the deeper layers of the skin. P. acnes, for example, resides around the hair follicles in high concentrations (10⁵ organisms per follicle) (Funke, G., et al., Clinical Microbiology Reviews, 10(1), 125-159 (1997)). HAI associated with these microorganisms arise due to the poor-efficacy and penetration of current skin disinfection techniques which kill microorganisms on the surface of the skin but fail to reach those residing in the deeper skin layers and around hair follicles. Furthermore microorganisms may become resistant to topical skin disinfection as they are protected by the follicles and surrounding lipids.

As a consequence, there are compelling health and economic grounds to establish novel disinfectants with more effective means of achieving skin disinfection.

Catheter-Related Infections

In the UK approximately 200,000 central venous catheters (CVC) are used annually for the intensive management of critically ill patients. The reported infection rate associated with CVC use is between 3 and 15%, with the Gram-positive coagulase negative staphylococci accounting for the majority of cases (Elliott, T. S. J., Catheter-associated infections: new developments in prevention. In: Burchardi H (ed). Current Topics in Intensive Care (volume 4). WB Saunders, London, 182-205 (1997)). Every year, almost 6000 patients in the UK acquire a catheter-related bloodstream infection (Fletcher, S. J. & Bodenham, A. R., British Journal of Intensive Care, 9, 46-53 (1999)) The costs associated with the treatment of CVC-sepsis are estimated to be ±2.5 million for long term catheters and ±5-7 million for short term (Moss, H. A. & Elliott, T. S. J., British Journal of Medical Economics, 11, 1-7 (1997)). Because of the intrinsic antibiotic resistance of CNS, vancomycin is widely used to treat CVC-sepsis, leading to a strong selective pressure for the emergence of vancomycin resistant enterococci (VRE). Novel methods of prevention are urgently needed to reduce the number of CNS infections related to CVC use and the associated risk of developing antibiotic resistant enterococci and staphylococci.

Risk factors for catheter-related infections include: (a) the device—catheter material and design; (b) operation—insertion procedures and catheter care; (c) the patient—immunosuppression, malignancy, concurrent infection, TPN; (d) medical personnel—cross infection. Preventative measures for catheter related infections include: antibiotic or antimicrobial coating, antibiotic lock; disinfection of insertion site, strict barrier precautions; antibiotic prophylaxis; training and application of guidelines. Randomized clinical trials have suggested the use of CVCs impregnated with either chlorhexidine and silver sulphadiazine or minocycline and rifampicin reduces the frequency of catheter related infections (Maki, D. G., et al., Ann Intern Med, 127, 257-266 (1997); Raad, I., et al. Ann Intern Med, 127, 267-274 (1997)). However resistance to chlorhexidine has been reported (Tattawasart, U., et al., J Hosp Infect, 42, 219-229 (1999)) and the overuse of CVC coated with rifampicin/minocycline may lead to development of antibiotic resistance amongst bacteria. Therefore, novel antimicrobial agents with Gram-positive activity which may be coated onto or impregnated into catheters are needed.

In a first aspect, the present invention resides in the use of compounds of formula (I), or salts or solvates thereof, as inhibitors of growth of Gram-positive bacteria, 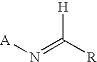
where A is selected from: 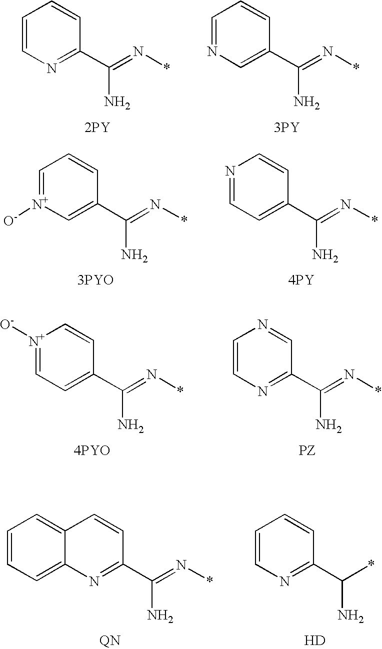
and
R is selected from optionally substituted C₅₋₂₀ aryl, with the proviso that when A is 2PY, then R is not 1,3-dimethylphenyl.

The use of compounds of formula (I) as inhibitors of growth of Gram-positive bacteria may or may not involve treatment of the human or animal body. When the use does not involve the treatment of the human or animal body, it may be termed in vitro, i.e. reproduction of a biological process in a more easily defined environment, and in particular, a culture vessel or plate.

Representative examples of gram-positive bacteria include Staphylococci (e.g. S. aureus, S. epidermis), Enterococci (e.g. E. faecium, E. faecalis), Clostridia (e.g. C. difficile), Propionibacteria (e.g. P. acnes) and Streptococci. Surprisingly, the inventors have found that some of the compounds of formula (I) exhibit inhibitory activity against bacterial strains resistant to other anti-bacterial agents such as methicillin and other isoxazolyl semisynthetic agents such as flucloxacillin, cloxacillin and oxacillin and glycopeptide antibiotics such as vancomycin. Some of the compounds exhibit a broad spectrum of activity against gram-positive bacteria.

As the inventors have found that some of the compounds of formula (I) have antimicrobial activity against a wide range of Gram positive microorganisms including multiple strains of MRSA and spore forming bacteria including Bacillus species, these compounds could be used to eliminate vegetative cells and spores of C. difficile in vitro, as shown in Example 3. The compounds of formula (I) may be used as surface disinfectants.

The potent Gram-positive antimicrobial activity of some of the compounds of the present invention make these compounds potentially suitable as surface disinfectants which may extend to the skin. Furthermore, the inherent lipophilic nature of the compounds of the present invention potentially makes them strong candidates for achieving effective skin penetration and disinfection of the deeper skin layers where many microorganisms reside and remain untouched by conventional current disinfectants.

For the avoidance of doubt, the Mycobacteria are not regarded for the purposes of this invention to be Gram-positive and therefore the use of the compounds of formula (I) as anti-Mycobacterial agents is outside the scope of the present invention.

Preferably, the compounds of the present invention selectively inhibit the growth of Gram-positive bacteria, e.g. are inactive against Gram-negative bacteria.

The general structure of the N¹-benzylideneheteroarylcarboxamidrazones is:—

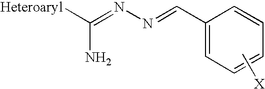

These compounds have proven to be of interest in the field of TB research; the antimycobacterial activities of a set of 2-pyridyl, 4-pyridyl and some 2-quinolylcarboxamidrazones have been examined and presented in a series of papers by Mamalo et al (e.g. Banfi, E., et al., J. Chemother. 5(3), 164-167 (1993)). From these works, the Mamalo group assimilated some qualitative structure-activity relationships. For their 2-pyridyl set of nineteen compounds, they found that there was a rough correlation between increased lipophilicity and improved mycobacterial inhibition. They found that compounds in which the arylmethylidene group possessed more polar substituents, such as methoxy, cyano or nitro groups, activity was either diminished or lost. Further work demonstrated that when the pyridine-based group was altered to 4-pyridyl, the activity approximately mirrored that of the 2-pyridyl compounds. The only 2-quinolylcarboxamidrazones for which the results are available are a small selection of 1-benzyl-1H-indol-3-ylidene derivatives. From these results, it was observed that the substitution of 2-pyridyl by 2-quinolyl resulted in a reduction of activity against mycobacteria. The most active compounds discovered by Mamalo et al included 2-chlorophenyl or 2-bromophenyl moieties, with both 2-pyridyl and 4-pyridyl-heteroaryl substituents, and some 1-benzyl-1H-indol-3-ylidene derivatives of 2-pyridylcarboxamidrazone.

Some copper complexes of the 2-pyridyl caboxamidrazones have been investigated for their anti-malarial and anti-cancer activity (Gokhale, N. H., et al., Inorg. Chim. Acta, 349, 23-29 (2003) and Gokhale, N. H., et al., Inorg. Chim. Acta, 319(1-2), 90-94 (2001)).

The hypertensive activity of certain 2-pyridyl carboxamidrazones has also been investigated (Vio, L., et al., Arch. Pharm., 321, 713-717 (1988)).

Interestingly, the inventors have found that certain of these compounds (and others previously not reported) have anti-microbial activity other than against mycobacteria, and that the pattern of activity is not predictable from the previously reported mycobacteria data. Of particular interest is the activity of some of the compounds against strains resistant to other agents. Although the anti-bacterial activity of certain 2- and 4-pyridyl carboxamidrazones has been investigated (Mamalo, et al., Eur. J. Med. Chim. Ther., 21(6), 467-474 (1986)), all but one were inactive against the panel bacteria.

The invention also resides in the use of a compound of formula (I), or a salt or solvate thereof, as an additive in selective culture medium or as a surface coating, particular on medical devices, such as catheters.

The invention further resides in the treatment of a human or animal patient afflicted with a Gram-positive bacterial infection, comprising administering to said patient an effective amount of a pharmaceutical composition containing a compound of formula (I), or a salt or solvate thereof. As discussed, in one aspect of the invention this application may be topical.

The invention yet further resides in the use of a compound of formula (I), or a salt or solvate thereof, in the manufacture of a medicament for the treatment of a Gram-positive bacterial infection in a human or other mammal. As discussed, in one aspect of the invention this medicament may be for topical administration.

Without wishing to be bound by theory, compounds of formula (I) where A is 3PYO and 4PYO are thought to be reduced to compounds of formula (I) where A is 3PY and 4PY respectively under bioreducing, e.g. hypoxic, conditions, which suggests their use as prodrugs in treating bioreducing, e.g. hypoxic, cancers.

The invention further resides in the following compounds or formula I, and salts or solvates thereof, where A and R are as defined above, unless otherwise stated:

(a) A is 3PYO or 4PYO;

(b) A is 3PY and R is optionally substituted C₅₋₂₀ carboaryl;

(c) A is 2PY, 3PY, 4PY, PZ, QN or HD and R is m-NO₂-phenyl, where the phenyl further bears a hydroxy substituent (which is preferably o-OH), and is optionally further substituted;

(d) A is 3PY or PY and R is 4-t-pentyl phenyl, where the phenyl is optionally further substituted;

(e) A is 2PY, 3PY, 4PY, PZ, QN or HD and R is trihydroxyphenyl;

(f) A is 2PY, 3PY, 4PY, PZ or QN and R is optionally further substituted dihydroxyphenyl;

(g) A is 2PY, 3PY, 4PY, PZ, QN or HD and R is p-OH phenyl where the phenyl bears a further hydroxy substituent, and is optionally further substituted;

(h) A is 2PY, 3PY, 4PY, PZ, QN or HD (preferably 4PY) and R is optionally substituted anthracenyl;

(i) A is 2PY, 3PY, 4PY, PZ, QN or HD (preferably 4PY) and R is 3-, 5-di-tbutyl phenyl, where the phenyl further bears a hydroxy substituent (which is preferably 2-OH);

(j) A is 4PY and R is thioether phenyl (preferably 2-thioether phenyl, and more preferably 2-thiophenyl); or

(k) A is HD and R is napthyl (preferably napth-1-yl, more preferably 2-hydroxynapth-1-yl).

The invention still further resides in the following compound:—

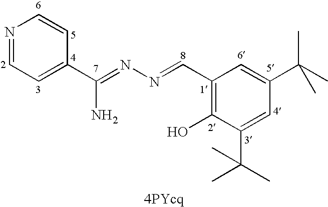

### DEFINITIONS

Aryl: The term “aryl”, as used herein, pertains to a monovalent moiety obtained by removing a hydrogen atom from an aromatic ring atom of an aromatic compound, which moiety has from 5 to 20 ring atoms (unless otherwise specified). Preferably, each ring has from 5 to 7 ring atoms.

In this context, the prefixes (e.g., C₅₋₂₀, C₅₋₇, C₅₋₆, etc.) denote the number of ring atoms, or range of number of ring atoms, whether carbon atoms or heteroatoms. For example, the term “C₅₋₆ aryl”, as used herein, pertains to an aryl group having 5 or 6 ring atoms. Examples of groups of aryl groups include C₅₋₂₀ aryl, C₅₋₁₅ aryl, C₅₋₁₂ aryl, C₅₋₁₀ aryl, C₅₋₇ aryl, C₅₋₆ aryl, C₅ aryl, and C₆ aryl.

The ring atoms may be all carbon atoms, as in “carboaryl groups.” Examples of carboaryl groups include C₅₋₂₀ carboaryl, C₅₋₁₅ carboaryl, C₅₋₁₂ carboaryl, C₅₋₁₀ carboaryl, C₅₋₇ carboaryl, C₅₋₆ carboaryl, C₅ carboaryl, and C₆ carboaryl.

Examples of carboaryl groups include, but are not limited to, those derived from benzene (i.e. phenyl) (C₆), naphthalene (C₁₀), azulene (C₁₀), anthracene (C₁₄), phenanthrene (C₁₄), naphthacene (C₁₈), and pyrene (C₁₆).

Examples of aryl groups which comprise fused rings, at least one of which is an aromatic ring, include, but are not limited to, groups derived from indane (e.g., 2,3-dihydro-1H-indene) (C₉), indene (C₉), isoindene (C₉), tetraline (1,2,3,4-tetrahydronaphthalene (C₁₀), acenaphthene (C₁₂), fluorene (C₁₃), phenalene (C₁₃), acephenanthrene (C₁₅), and aceanthrene (C₁₆).

Alternatively, the ring atoms may include one or more heteroatoms, as in “heteroaryl groups”. Examples of heteroaryl groups include C₅₋₂₀ heteroaryl, C₅₋₁₅ heteroaryl, C₅₋₁₂ heteroaryl, C₅₋₁₀ heteroaryl, C₅₋₇ heteroaryl, C₅₋₆ heteroaryl, C₅ heteroaryl, and C₆ heteroaryl.

Examples of monocyclic heteroaryl groups include, but are not limited to, those derived from:

N₁: pyrrole (azole) (C₅), pyridine (azine) (C₆);

O₁: furan (oxole) (C₅);

S₁: thiophene (thiole) (C₅);

N₁O₁: oxazole (C₅), isoxazole (C₅), isoxazine (C₆);

N₂O₁: oxadiazole (furazan) (C₅);

N₃O₁: oxatriazole (C₅);

N₁S₁: thiazole (C₅), isothiazole (C₅);

N₂: imidazole (1,3-diazole) (C₅), pyrazole (1,2-diazole) (C₅), pyridazine (1,2-diazine) (C₆), pyrimidine (1,3-diazine) (C₆) (e.g., cytosine, thymine, uracil), pyrazine (1,4-diazine) (C₆);

N₃: triazole (C₅), triazine (C₆); and,

N₄: tetrazole (C₅).

Examples of heteroaryl groups which comprise fused rings, include, but are not limited to:

C₉ heteroaryl groups (with 2 fused rings) derived from benzofuran (O₁), isobenzofuran (O₁), indole (N₁), isoindole (N₁), indolizine (N₁), indoline (N₁), isoindoline (N₁), purine (N₄) (e.g., adenine, guanine), benzimidazole (N₂), indazole (N₂), benzoxazole (N₁O₁), benzisoxazole (N₁O₁), benzodioxole (O₂), benzofurazan (N₂O₁), benzotriazole (N₃), benzothiofuran (S₁), benzothiazole (N₁S₁), benzothiadiazole (N₂S);

C₁₀heteroaryl groups (with 2 fused rings) derived from chromene (O₁), isochromene (O₁), chroman (O₁), isochroman (O₁), benzodioxan (O₂), quinoline (N₁), isoquinoline (N₁), quinolizine (N₁), benzoxazine (N₁O₁), benzodiazine (N₂), pyridopyridine (N₂), quinoxaline (N₂), quinazoline (N₂), cinnoline (N₂), phthalazine (N₂), naphthyridine (N₂), pteridine (N₄);

C₁₁ heteroaryl groups (with 2 fused rings) derived from benzodiazepine (N₂);

C₁₃ heteroaryl groups (with 3 fused rings) derived from carbazole (N₁), dibenzofuran (O₁), dibenzothiophene (S₁), carboline (N₂), perimidine (N₂), pyridoindole (N₂); and,

C₁₄ heteroaryl groups (with 3 fused rings) derived from acridine (N₁), xanthene (O₁), thioxanthene (S₁), oxanthrene (O₂), phenoxathiin (O₁S₁), phenazine (N₂), phenoxazine (N₁O₁), phenothiazine (N₁S₁), thianthrene (S₂), phenanthridine (N₁), phenanthroline (N₂), phenazine (N₂).

Cancers: Examples of cancers include, but are not limited to, lung cancer, small cell lung cancer, gastrointestinal cancer, bowel cancer, colon cancer, breast carinoma, ovarian carcinoma, prostate cancer, testicular cancer, liver cancer, kidney cancer, bladder cancer, pancreas cancer, brain cancer, sarcoma, osteosarcoma, Kaposi's sarcoma and melanoma.

Further Preferences

The following preferences may be combined with one another, where appropriate, and may apply to any relevant aspect of the present invention.

In some embodiments, preferably A is selected from 2PY, 4PY and HD, more preferably 4PY and HD and most preferably 4PY.

R may be selected from the group consisting of: phenyl, naphthyl (e.g. naphth-1-yl or naphth-2-yl), anthryl (e.g. 9-anthryl), phenanthryl (e.g. 9-phenanthryl), pyrrolyl (e.g. pyrrol-2-yl), imidazolyl, pyridinyl (e.g. pyridin-2-yl or pyridin-3-yl), furanyl, thiophenyl, quinolinyl, 1,4-benzopyronyl (e.g. 1,4-benzopyron-3-yl), pyrazolyl, isoxazolyl, oxazolyl, thiazolyl, pyridazinyl, pyrimidinyl, pyrazinyl, indolyl, isoindolyl, indazolyl, indolizidinyl, isoquinolinyl and quinazolinyl.

It may be preferred that R is not 1,3-dimethyl phenyl.

Preferably, R is substituted phenyl, substituted 1-naphthyl or 9-anthryl (substituted or unsubstituted) and most preferably substituted phenyl.

When R is substituted, said substituent or substituents is/are preferably selected from hydroxy, C₁₋₆ straight, branched or cyclic alkyl (e.g. Me, CF₃), C₁₋₆ straight, branched or cyclic alkoxy or alkylthio, C₁₋₆ straight, branched or cyclic alkylcarbonyloxy, carboxy (CO₂H), C₁₋₆ straight, branched or cyclic alkyloxycarbonyl, C₁₋₆ straight, branched or cyclic alkylcarbonylamino, cyano (CN), amino (e.g. NR^{N1}R^{N2}, where R^{N1} and R^{N2} are independently selected from H and C₁₋₆ alkyl, or together with the nitrogen to which they are attached form a 3 to 7 membered heterocyclic ring, e.g. piperadinyl, piperazinyl, morpholino), nitro (NO₂), amido (e.g. C(═O)NR^{N1}R^{N2}, wherein R^{N1} and R^{N2} are as for amino), halo (e.g. F, Cl, Br, I), C₅₋₂₀ aryl (e.g. phenyl), benzyl or phenyl(C₁₋₆)alkyloxy, wherein each C₁₋₆ alkyl or C₅₋₂₀ aryl group (e.g. phenyl) being substituted or unsubstituted. In some embodiments, the C₁₋₆ alkyl groups are unsubstituted.

Preferred substituents are hydroxy, methoxy, ^{t}butyl, 1,1-dimethylpropyl, phenylthio (itself substituted or unsubstituted), aminoalkyloxy, iodo, bromo and nitro.

Preferably, R is at least di-substituted (especially when R is phenyl), one of said substituents preferably being hydroxy or ^{t}butyl. 2-hydroxy substituted derivatives of R are especially preferred.

Specific examples of

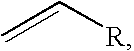

particularly for use where A is 2PY, 3PY, 4PY, PZ, QN and HD, are shown in Table 1 below.

TABLE 1

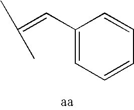

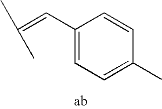

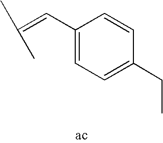

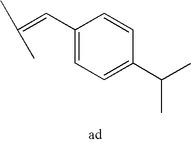

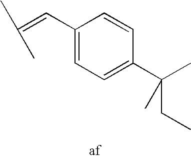

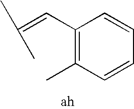

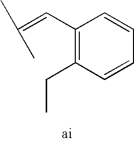

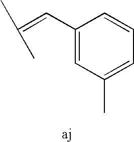

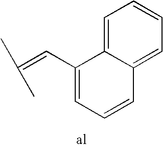

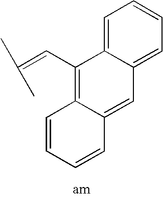

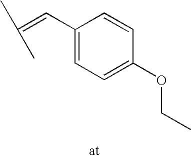

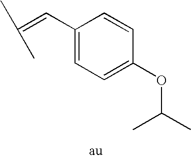

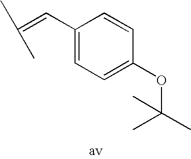

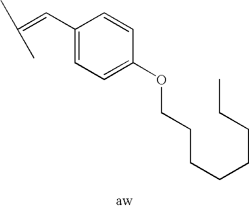

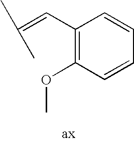

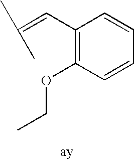

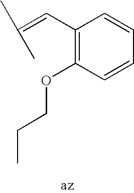

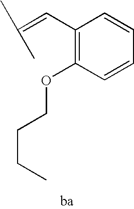

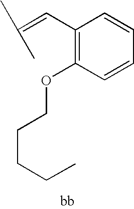

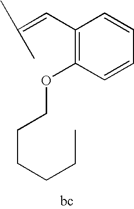

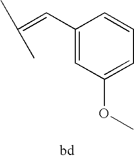

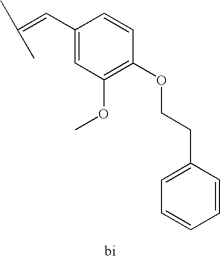

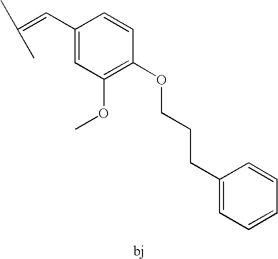

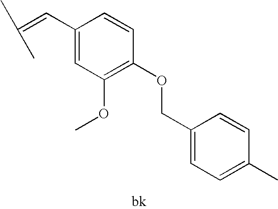

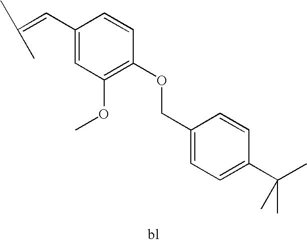

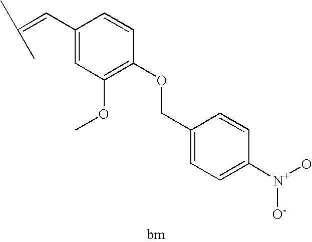

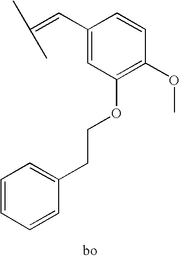

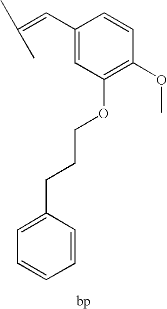

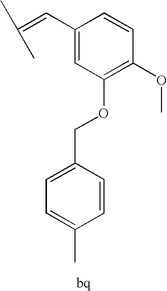

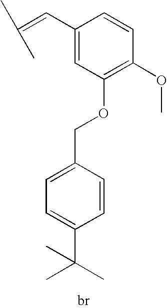

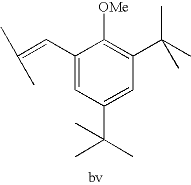

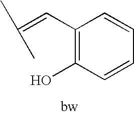

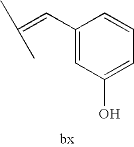

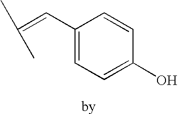

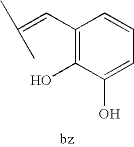

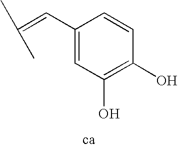

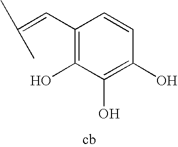

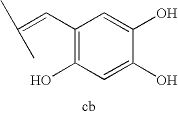

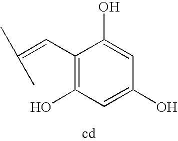

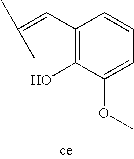

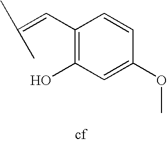

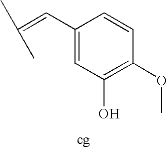

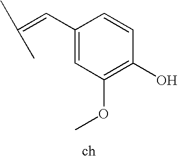

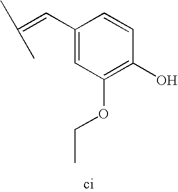

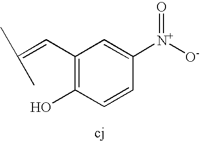

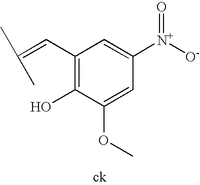

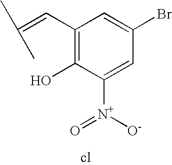

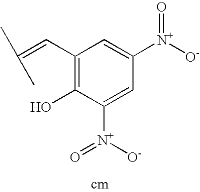

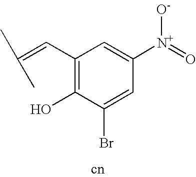

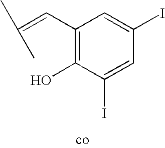

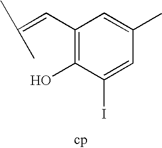

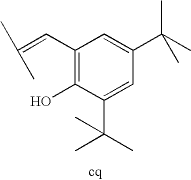

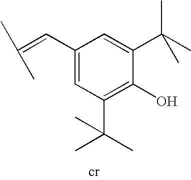

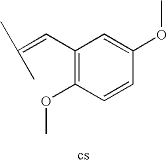

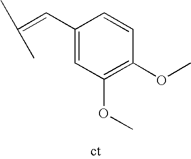

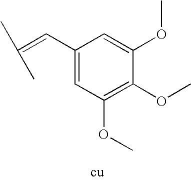

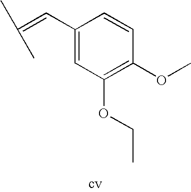

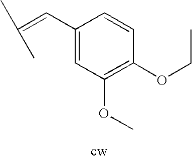

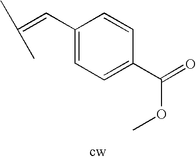

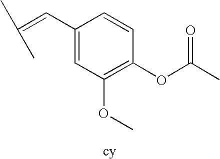

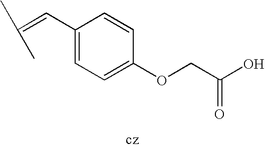

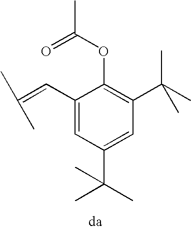

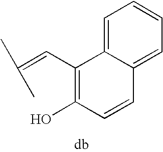

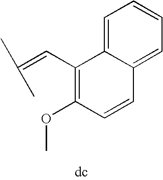

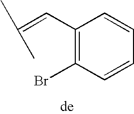

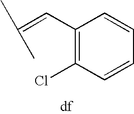

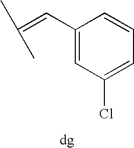

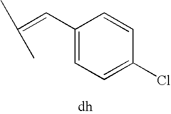

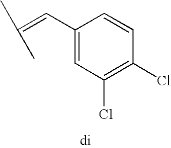

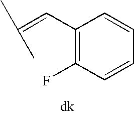

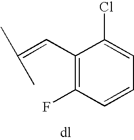

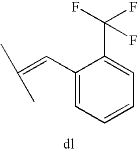

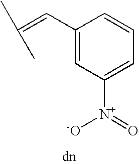

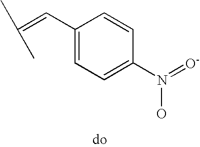

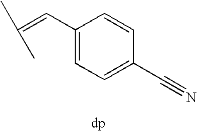

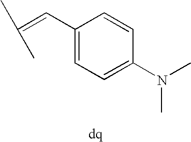

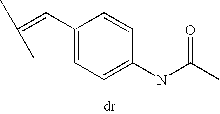

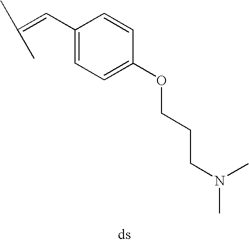

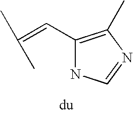

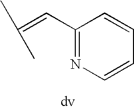

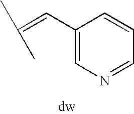

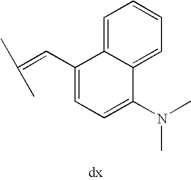

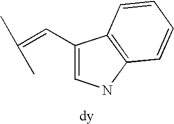

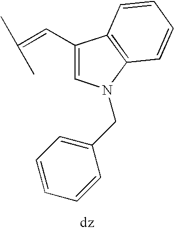

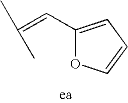

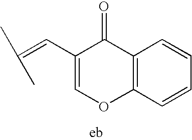

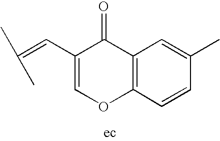

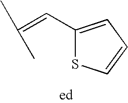

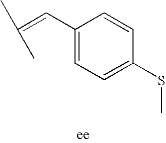

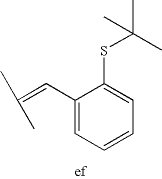

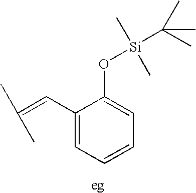

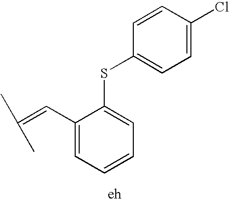

When A is 2PY, R is preferably af, ah, ai, aj, al or cj.

When A is 3PY, R is preferably af, ay, cc, cj or cl.

When A is 4PY, R is preferably, af, am, cb, cc, cj or co.

When A is HD, R is preferably cd, ce, cf, cj or cl.

When A is PZ, R is preferably, cb or cj.

When A is QN, R is preferably ca.

Particularly preferred compounds are 3PYaf, 4PYaf, 4PYam, 4PYcb, 4PYco, 4PYcq, 4PYeh, HDcb, HDce, HDcf and HDdb. The most preferred compound is 4PYcq.

Specific examples of

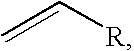

particularly for use where A is 3PYO and 4PYO, are shown in Table 2 below.

TABLE 2

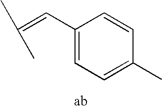

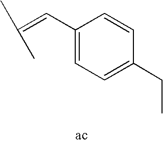

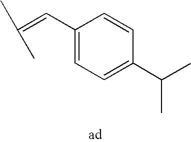

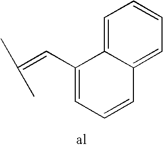

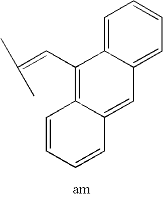

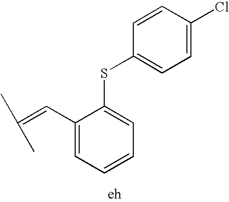

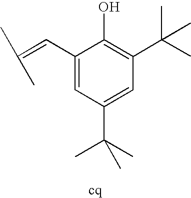

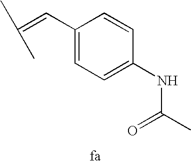

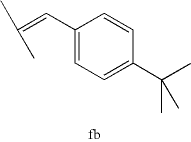

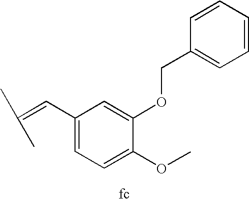

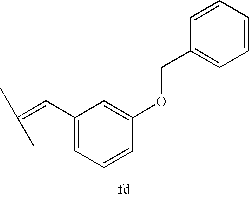

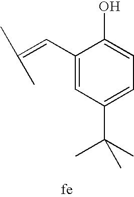

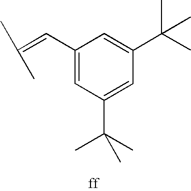

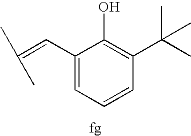

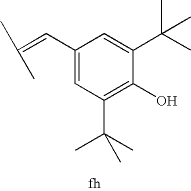

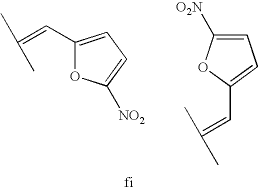

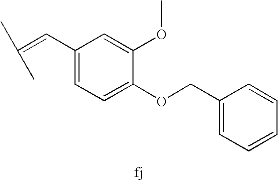

When A is 4PYO, R is preferably cq. Accordingly, a particularly preferred compound is 4PYOcq.

Isomers, Salts and Solvates

Isomers

Certain compounds may exist in one or more particular geometric, optical, enantiomeric, diasteriomeric, epimeric, atropic, stereoisomeric, tautomeric, conformational, or anomeric forms, including but not limited to, cis- and trans-forms; E- and Z-forms; c-, t-, and r-forms; endo- and exo-forms; R-, S-, and meso-forms; D- and L-forms; d- and l-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

Note that, except as discussed below for tautomeric forms, specifically excluded from the term “isomers”, as used herein, are structural (or constitutional) isomers (i.e. isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g., C₁₋₇alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol (illustrated below), imine/enamine, amide/imino alcohol, amidine/amidine, nitroso/oxime, thioketone/enethiol, N-nitroso/hydroxyazo, and nitro/aci-nitro.

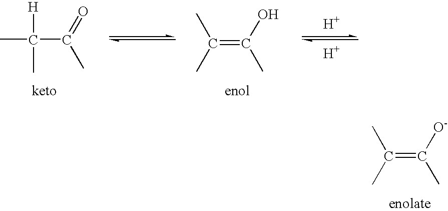

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Unless otherwise specified, a reference to a particular compound includes all such isomeric forms, including (wholly or partially) racemic and other mixtures thereof. Methods for the preparation (e.g. asymmetric synthesis) and separation (e.g. fractional crystallisation and chromatographic means) of such isomeric forms are either known in the art or are readily obtained by adapting the methods taught herein, or known methods, in a known manner.

Salts

It may be convenient or desirable to prepare, purify, and/or handle a corresponding salt of the active compound, for example, a pharmaceutically-acceptable salt. Examples of pharmaceutically acceptable salts are discussed in Berge, et al., J. Pharm. Sci., 66, 1-19 (1977).

For example, if the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al⁺³. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group which may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous.

Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, and valeric. Examples of suitable polymeric organic anions include, but are not limited to, those derived from the following polymeric acids: tannic acid, carboxymethyl cellulose.

Unless otherwise specified, a reference to a particular compound also include salt forms thereof.

Solvates

It may be convenient or desirable to prepare, purify, and/or handle a corresponding solvate of the active compound. The term “solvate” is used herein in the conventional sense to refer to a complex of solute (e.g., active compound, salt of active compound) and solvent. If the solvent is water, the solvate may be conveniently referred to as a hydrate, for example, a mono-hydrate, a di-hydrate, a tri-hydrate, etc.

Unless otherwise specified, a reference to a particular compound also include solvate forms thereof.

Dosage and Formulation

The dosage administered to a patient will normally be determined by the prescribing physician and will generally vary according to the age, weight and response of the individual patient, as well as the severity of the patient's symptoms and the proposed route of administration. However, in most instances, an effective therapeutic daily dosage will be in the range of from about 0.05 mg/kg to about 100 mg/kg of body weight and, preferably, of from 0.5 mg/kg to about 20 mg/kg of body weight administered in single or divided doses. In some cases, however, it may be necessary to use dosages outside these limits.

While it is possible for an active ingredient to be administered alone as the raw chemical, it is preferable to present it as a pharmaceutical formulation. The formulations, both for veterinary and for human medical use, of the present invention comprise a compound of formula (I) in association with a pharmaceutically acceptable carrier therefor and optionally other therapeutic ingredient(s). The carrier(s) must be ‘acceptable’ in the sense of being compatible with the other ingredients of the formulation and not deleterious to the recipient thereof.

Conveniently, unit doses of a formulation contain between 0.1 mg and 1 g of the active ingredient. Preferably, the formulation is suitable for administration from one to six, such as two to four, times per day. For topical administration, the active ingredient preferably comprises from 1% to 2% by weight of the formulation but the active ingredient may comprise as much as 10% w/w. Formulations suitable for nasal or buccal administration, such as the self-propelling powder-dispensing formulations described hereinafter, may comprise 0.1 to 20% w/w, for example about 2% w/w of active ingredient.

The formulations include those in a form suitable for oral, ophthalmic, rectal, parenteral (including subcutaneous, vaginal, intraperitoneal, intramuscular and intravenous), intraarticular, topical, nasal or buccal administration. The toxicity of certain of the compounds in accordance with the present invention will preclude their administration by systemic routes, and in those, and other, cases opthalmic, topical or buccal administration, and in particular topical administration, is preferred for the treatment of local infection.

Formulations of the present invention suitable for oral administration may be in the form of discrete units such as capsules, cachets, tablets or lozenges, each containing a predetermined amount of the active ingredient; in the form of a powder or granules; in the form of a solution or a suspension in an aqueous liquid or non-aqueous liquid; or in the form of an oil-in-water emulsion or a water-in-oil emulsion. The active ingredient may also be in the form of a bolus, electuary or paste. For such formulations, a range of dilutions of the active ingredient in the vehicle is suitable, such as from 1% to 99%, preferably 5% to 50% and more preferably 10% to 25% dilution.

Formulations for rectal administration may be in the form of a suppository incorporating the active ingredient and a carrier such as cocoa butter, or in the form of an enema.

Formulations suitable for parenteral administration comprise a solution, suspension or emulsion, as described above, conveniently a sterile aqueous preparation of the active ingredient that is preferably isotonic with the blood of the recipient.

Formulations suitable for intra-articular administration may be in the form of a sterile aqueous preparation of the active ingredient, which may be in a microcrystalline form, for example, in the form of an aqueous microcrystalline suspension or as a micellar dispersion or suspension. Liposomal formulations or biodegradable polymer systems may also be used to present the active ingredient particularly for both intra-articular and ophthalmic administration.

Formulations suitable for topical administration include liquid or semi-liquid preparations such as liniments, lotions or applications; oil-in-water or water-in-oil emulsions such as creams, ointments or pastes; or solutions or suspensions such as drops. For example, for ophthalmic administration, the active ingredient may be presented in the form of aqueous eye drops, as for example, a 0.1-1.0% solution.

Drops according to the present invention may comprise sterile aqueous or oily solutions. Preservatives, bactericidal and fungicidal agents suitable for inclusion in the drops are phenylmercuric salts (0.002%), benzalkonium chloride (0.01%) and chlorhexidine acetate (0.01%). Suitable solvents for the preparation of an oily solution include glycerol, diluted alcohol and propylene glycol.

Lotions according to the present invention include those suitable for application to the eye. An eye lotion may comprise a sterile aqueous solution optionally containing a bactericide or preservative prepared by methods similar to those for the preparation of drops. Lotions or liniments for application to the skin may also include an agent to hasten drying and to cool the skin, such as an alcohol, or a softener or moisturiser such as glycerol or an oil such as castor oil or arachis oil.

Creams, ointments or pastes according to the present invention are semi-solid formulations of the active ingredient in a base for external application. The base may comprise one or more of a hard, soft or liquid paraffin, glycerol, beeswax, a metallic soap; a mucilage; an oil such as a vegetable oil, eg almond, corn, arachis, castor or olive oil; wool fat or its derivatives; or a fatty acid ester of a fatty acid together with an alcohol such as propylene glycol or macrogols. The formulation may also comprise a suitable surface-active agent, such as an anionic, cationic or non-ionic surfactant such as a glycol or polyoxyethylene derivatives thereof. Suspending agents such as natural gums may be incorporated, optionally with other inorganic materials, such as silicaceous silicas, and other ingredients such as lanolin.

Formulations suitable for administration to the nose or buccal cavity include those suitable for inhalation or insufflation, and include powder, self-propelling and spray formulations such as aerosols and atomisers. The formulations, when dispersed, preferably have a particle size in the range of 10 to 200μ.

Such formulations may be in the form of a finely comminuted powder for pulmonary administration from a powder inhalation device or self-propelling powder-dispensing formulations, where the active ingredient, as a finely comminuted powder, may comprise up to 99.9% w/w of the formulation.

Self-propelling powder-dispensing formulations preferably comprise dispersed particles of solid active ingredient, and a liquid propellant having a boiling point of below 18° C. at atmospheric pressure. Generally, the propellant constitutes 50 to 99.9% w/w of the formulation whilst the active ingredient constitutes 0.1 to 20% w/w. for example, about 2% w/w, of the formulation.

The pharmaceutically acceptable carrier in such self-propelling formulations may include other constituents in addition to the propellant, in particular a surfactant or a solid diluent or both. Especially valuable are liquid non-ionic surfactants and solid anionic surfactants or mixtures thereof. The liquid non-ionic surfactant may constitute from 0.01 up to 20% w/w of the formulation, though preferably it constitutes below 1% w/w of the formulation. The solid anionic surfactants may constitute from 0.01 up to 20% w/w of the formulation, though preferably below 1% w/w of the composition.

Formulations of the present invention may also be in the form of a self-propelling formulation wherein the active ingredient is present in solution. Such self-propelling formulations may comprise the active ingredient, propellant and co-solvent, and advantageously an antioxidant stabiliser. Suitable co-solvents are lower alkyl alcohols and mixtures thereof. The co-solvent may constitute 5 to 40% w/w of the formulation, though preferably less than 20% w/w of the formulation. Antioxidant stabilisers may be incorporated in such solution-formulations to inhibit deterioration of the active ingredient and are conveniently alkali metal ascorbates or bisulphites. They are preferably present in an amount of up to 0.25% w/w of the formulation.

Formulations of the present invention may also be in the form of an aqueous or dilute alcoholic solution, optionally a sterile solution, of the active ingredient for use in a nebuliser or atomiser, wherein an accelerated air stream is used to produce a fine mist consisting of small droplets of the solution.

In addition to the aforementioned ingredients, the formulations of this invention may include one or more additional ingredients such as diluents, buffers, flavouring agents, binders, surface active agents, thickeners, lubricants, preservatives eg methylhydroxybenzoate (including anti-oxidants), emulsifying agents and the like. A particularly preferred carrier or diluent for use in the formulations of this invention is a lower alkyl ester of a C₁₈ to C₂₄ mono-unsaturated fatty acid, such as oleic acid, for example ethyl oleate. Other suitable carriers or diluents include capric or caprylic esters or triglycerides, or mixtures thereof, such as those caprylic/capric triglycerides sold under the trade name Miglyol, eg Miglyol 810.

Embodiments of the invention will now be described by way of example only.

The preparation of the N¹-benzylideneheteroarylcarboxamidrazones and N¹-benzylideneheteroarylcarboxamidrazones-N-oxides of the present invention is shown in Scheme 1 below.

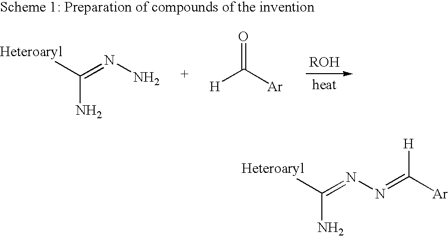

### EXAMPLE 1

### Synthesis of N¹-benzylideneheteroarylcarboxamidrazones

A large variety of aldehydes were investigated and are referred to throughout by a two lower-case letter code. The structures of the aldehyde residues are shown in Table 1 above and are referenced by the same two letter code for convenience.

Since a large library of compounds was to be produced robotically, it was necessary to probe the versatility of the reaction, to see if it could cope with aldehydes possessing different electronic and steric properties. Initially, some 4-substituted benzaldehydes with differing electronic natures were chosen to investigate the reaction. For example, 4-isopropylbenzaldehyde ad and 4-hydroxybenzaldehyde by, were used to represent aldehydes with electron-donating groups, whilst 4-chlorobenzaldehyde dh and 4-cyanobenzaldehyde represented electron-withdrawing substituents. The 4-position was favoured at this developmental stage due to the ease of analysis in ¹H NMR.

Steric factors were then taken into consideration. For example, would bulky substituents in either one or both of the ortho positions of the benzaldehyde hinder the reaction? For this, 2-ethylbenz-aldehyde ai, the 2-alkyloxybenzaldehydes ay-bc, and the disubstituted 2-,5-dichloro-benzaldehyde dj, were used to investigate. 2-trifluoromethylbenzaldehyde dm was used to test a very bulky electron-withdrawing substituent, and bulky silyl-ether eg, was used to test a very steric, strongly electron-donating one.

Pyridine-2-carboxamidrazone 2PY was reacted with all these ‘test’ aldehydes and the products analysed by ¹H NMR, which showed that all the reactions proceeded to give the desired products. From this, it was established that the reaction was very versatile, and that almost any aromatic aldehyde could be used.

Most of the aldehydes used in this work were commercially available, some were prepared in the laboratory (bi, bj, bk, bl, bp, bq, br, cp, da) and a few were prepared by a previous workers using standard literature procedures (az, ba, bb, bc, bo).

The heteroarylcarboxamidrazones building blocks 2PY, 3PY, 4PY, PZ, QN were prepared by the action of hydrazine (hydrazine hydrate for 2PY, PZ, QN and 80% hydrazine for 3PY and 4PY) upon the corresponding cyano compounds.

N¹-Benzylideneheteroarylcarboxamidrazone Library Synthesis

An initial library of the condensation products of heteroaryl-carboxamidrazones and aldehydes was prepared using automated parallel solution phase synthesis. A robotic pipetting station was used to transfer stock solutions of previously synthesised heteroarylcarboxamidrazones in methanol, and stock solutions of aldehydes in ethanol, into a matrix of 90 empty 4 ml vials. A heating block was used to heat the matrix of reactions at reflux for an appropriate period. Upon cooling, most of the products precipitated out of solution, and a crude work-up was effected by automation to remove the soluble excess starting materials and by-products. Ethanol was transferred by pipette, into the product vials, allowed to stand, and then removed: a process known as trituration, which was repeated twice more. For the more soluble products which dissolved in ethanol, either ether or petroleum ether was used to wash the compounds instead, in order to increase the product recovery. Within each matrix of 90 vials, the separate vials contained only one heteroarylcarboxamidrazone and only one aldehyde building block, to give one product per vial.

The product compound codes are such that if pyridine-2-carboxamidrazone 2PY, is reacted with benzaldehyde aa, then the product is called 2PYaa. The capital letters refer to the amidrazone or hydrazone moiety and the two lower-case letters refer to the aldehyde-derived substituent.

The compounds 2PYaa, 2PYab, 2PYax, 2PYde, 2PYdf, 2PYdh, 2PYdi, 2PYdo, 2PYdp and 4PYaa, 4PYab, 4PYax, 4PYde, 4PYdf, 4PYdh, 4PYdi, 4PYdo, 4PYdp have been reported previously by Mamalo et al.

All compounds were characterised by positive atmospheric pressure ionisation mass spectrometry (APCI-MS) and all exhibited a dominant (M+H)⁺ peak. Prior to biological testing, at least 10% of the compounds were analysed by ¹H NMR, which confirmed the structures, with purity generally greater than 85%, and often greater than 95%. The only impurities generally detected in the NMR spectra were excess aldehyde, except in the case of the pyridine-3-carboxamidrazones, where a side reaction occurred. Thin layer chromatography of all the compounds also showed the same trend, where only one spot was usually seen, unless some unreacted aldehyde remained, or, for pyridine-3-carboxamidrazones, a by-product was produced.

The high purity of these products may be somewhat surprising since it has been reported that amidrazones can self-condense at elevated temperatures. This potential side reaction, however, was not observed, except perhaps in the case of pyridine-3-carboxamidrazones, where bis-hydrazones were isolated. The fact that this side reaction was not generally observed may be due to the fact that an excess of aldehyde was used, and that the reaction components were assembled at ambient temperature before being heated up, relatively slowly, to the boiling point of methanol. It is likely that this operation, combined with precipitation of the benzylidene products, favoured benzylidene formation over the competing self-condensation reaction pathway.

Several compounds exhibited promising activity against the organisms tested. 4PYcq was the lead compound with the most interesting activity against S. aureus, E. faecium and MRSA. Some analogues of aldehyde cq were synthesised (See Table 2) to investigate the effect of various molecular alterations.

TABLE 2
Substituents derived from the aldehydes synthesised to further
investigate the antibacterial activity of 4PYcq

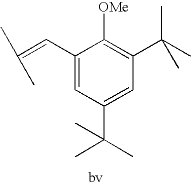

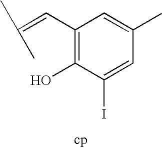

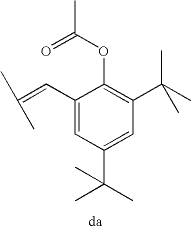

Aldehydes bv and da were both derived from aldehyde cq. Alkylation of the hydroxyl group of cq, by methyl iodide gave bv, and acetylation of the same hydroxyl group using acetic anhydride gave da. bv was synthesised to investigate the importance of the hydroxyl group of cq, and da was prepared for much the same reason, although it is possible that hydrolysis of the acetyl group of this molecule could occur in vivo, to give the original compound.

Aldehyde cp was prepared from 2,4-dimethylphenol and paraformaldehyde according to the method proposed by Casiraghi et al “Selective reactions between phenols and formaldehyde. A novel route to salicylaldehydes.” J. Chem. Soc. Perkin Trans. 1 (1980), pp 1862-1865. This aldehyde replaces the t-butyl groups of cq with less lipophillic methyl groups.

Anti-Bacterial Activity

Antibacterial testing results of selected heteroarylbenzylidene carboxamidrazones are shown in Table 3 below.

This activity was measured using a multipoint inoculator method as follows. The MIC for each compound was measured using an agar dilution method (Onda, H., et al., Int. J. Antimicrob. Agents. 18, 263 (2001)) (Mueller Hinton agar) by means of a multipoint inoculator delivering 10⁴ colony forming units per spot. The MIC was defined as the lowest concentration inhibiting growth after incubation at 37° C. for 18 hours.

‘Staph’ refers to the reference strain of S. aureus (NCTC 6571). A tick in the MRSA column refers to a positive zone against MRSA strain 96-7474, and the MIC range (in μg/ml) which follows is that found against a panel of ten MRSA strains. Where MIC values are given for MRSA, these are stated as a range of values, as testing was carried out on a panel of clinical isolates.

| | Staph | MRSA |
|---|---|---|
| 2PYab | ✓ | X |
| 2PYaf | ✓ | ✓>256 |
| 2PYah | ✓ | ✓128-256 |
| 2PYai | ✓ | ✓128-256 |
| 2PYaj | ✓ | ✓128-256 |
| 2PYal | ✓ | ✓>256 |
| 2PYca | ✓ | X |
| 2PYcb | ✓ | ✓ |
| 2PYcc | ✓ | X |
| 2PYcf | X | ✓ |
| 2PYcj | ✓ | ✓4-32 |
| 2PYcl | ✓ | ✓ |
| 2PYcm | ✓ | ✓ |
| 2PYdb | ✓ | ✓>256 |
| 2PYdg | ✓ | ✓64-128 |
| 2PYdh | ✓ | X |
| 2PYdk | ✓ | X |
| 2PYdm | ✓ | X |
| 2PYdq | ✓ | ✓128-256 |
| 2PYeb | ✓ | X |
| 2PYeh | ✓ | X |
| 3PYaf | ✓ | ✓32-64 |
| 2PYal | ✓ | X |
| 3PYay | ✓ | ✓>256 |
| 3PYcb | ✓ | X |
| 3PYcc | ✓ | ✓128-256 |
| 3PYcj | ✓ | ✓2-8 |
| 3PYcl | ✓ | ✓16-64 |
| 3PYdb | ✓ | ✓128-256 |
| 4PYaf | ✓ | ✓32-64 |
| 4PYam | ✓ | ✓16-64 |
| 4PYcb | ✓ | ✓30-40 |
| 4PYcc | ✓ | ✓20-30 |
| 4PYcj | ✓ | ✓20-30 |
| 4PYcl | ✓ | X |
| 4PYcn | ✓ | X |
| 4PYco | ✓ | ✓10-20 |
| 4PYcq | ✓ | ✓2-4 |
| 4PYcr | ✓ | ✓>256 |
| 4PYdb | ✓ | X |
| 4PYdx | ✓ | ✓64-128 |
| 4PYeh | ✓ | ✓8-64 |
| HDbz | ✓ | X |
| HDcb | ✓ | ✓40-60 |
| HDcc | ✓ | X |
| HDcd | X | ✓128-256 |
| HDce | ✓ | ✓10-20 |
| HDcf | ✓ | ✓20-30 |
| HDcj | ✓ | ✓16-64 |
| HDcl | ✓ | ✓64-128 |
| HDdb | ✓ | ✓4-16 |
| HDdv | ✓ | ✓ |
| PZca | ✓ | X |
| PZcb | ✓ | ✓128-256 |
| PZcc | ✓ | ✓ |
| PZcj | ✓ | ✓4-16 |
| PZdp | ✓ | X |
| PZeg | ✓ | ✓>256 |
| QNca | ✓ | ✓64-128 |
| QNcb | ✓ | X |
| QNcc | ✓ | ✓ |
| QNds | ✓ | ✓32-128 |
| | X | X |

Table 4 shows the broad spectrum activity of some of the compounds, wherein the MICS were determined by the multipoint inoculator method described above. Fluclox.=Flucloxacillin, Amp.=Ampicillin, Vanc.=Vancomycin hydrochloride. Upper values of the MIC readings are given.

| | Reference | | | | | | | | | | | |
|---|---|---|---|---|---|---|---|---|---|---|---|---|
| | | | | Iso- | Iso- | Iso- | Iso- | Iso- | Iso- | Iso- | Iso- | Iso- |
| | NCTC 6571 | NCTC 10788 | Cowan 1 | late 1 | late 2 | late 3 | late 4 | late 5 | late 6 | late 7 | late 8 | late 9 |
| | Strain | | | | | | | | | | | |
| Code | S. aureus | S. aureus | S. aureus | MRSA | MRSA | MRSA | MRSA | MRSA | MRSA | MRSA | MRSA | MRSA |
| Fluclox. | 0.05 | 0.05 | 0.05 | 0.05 | 8 | 8 | 16 | 0.05 | 0.25 | 0.25 | 8 | 8 |
| Amp. | 0.05 | 0.05 | 0.05 | 0.12 | 8 | 8 | 8 | 8 | 8 | 8 | 8 | 8 |
| Vanc. | 0.05 | 0.12 | 0.25 | 0.12 | 0.12 | 0.12 | 0.12 | 0.12 | 0.12 | 0.12 | 0.12 | 0.12 |
| 3PYaf | 64 | 64 | 64 | 64 | 64 | 64 | 64 | 64 | 64 | 64 | 64 | 64 |
| 4PYaf | 64 | 64 | 64 | 64 | 64 | 64 | 64 | 64 | 64 | 64 | 64 | 64 |
| 4PYam | 32 | 64 | 32 | 32 | 64 | 64 | 32 | 32 | 32 | 64 | 64 | 32 |
| 3PYay | >256 | X | X | 256 | 128 | 128 | 128 | 128 | X | X | X | X |
| QNca | 128 | 128 | 128 | 128 | 128 | 128 | 128 | 128 | 128 | 128 | 128 | 128 |
| 2PYcb | X | X | 256 | X | X | X | X | 256 | X | X | X | X |
| PZcb | 256 | 256 | 256 | 256 | 256 | 256 | 256 | 256 | 256 | 256 | 256 | 256 |
| 3PYcc | 256 | 256 | 256 | 256 | 256 | 256 | 256 | 256 | 256 | 256 | 256 | 256 |
| QNcc | 256 | 256 | 256 | 256 | X | X | X | X | X | X | X | X |
| HDcd | 256 | 256 | 256 | 256 | 256 | 256 | 256 | 256 | 256 | 256 | 256 | 256 |
| cj | 4 | 2 | 16 | 2 | 4 | 2 | 2 | 2 | 4 | 4 | 4 | 4 |
| 2PYcj | 16 | 16 | 32 | 16 | 16 | 4 | 8 | 16 | 16 | 8 | 4-8 | 8 |
| 3PYcj | 8 | 8 | 16 | 4 | 4 | 4 | 4 | 4 | 8 | 8 | 8 | 8 |
| PZcj | 16 | 16 | 256 | 8 | 8 | 4 | 4 | 16 | 16 | 16 | 16 | 16 |
| HDcj | 32 | 32 | 32 | 64 | 64 | 64 | 32 | 32 | 64 | 64 | 64 | 64 |
| cl | 16 | 16 | 32 | 16 | 16 | 32 | 16 | 16 | 32 | 32 | 32 | 32 |
| 2PYcl | 32 | 32 | 64 | 64 | 64 | 64 | 32 | 32 | 64 | 64 | 64 | 64 |
| 3PYcl | 32 | 32 | 64 | 64 | 64 | 64 | 32 | 32 | 64 | 64 | 64 | 64 |
| QNcl | >256 | X | X | X | 64 | 64 | 64 | X | X | X | X | X |
| HDcl | 128 | 128 | 128 | 128 | 128 | 128 | 128 | 128 | 128 | 128 | 128 | 128 |
| 4PYcq | 4 | 4 | 4 | 4 | 4 | 4 | 4 | 4 | 4 | 4 | 4 | 4 |
| 4PYcr | >256 | X | X | X | X | >256 | X | X | X | X | X | X |
| 2PYdb | >256 | X | X | X | X | >256 | X | X | X | X | X | X |
| 3PYdb | 256 | 256 | 256 | 256 | 256 | 256 | 256 | 256 | 256 | 256 | 256 | 256 |
| 4PYdb | >256 | X | 128 | 128 | X | 256 | 256 | X | X | X | X | X |
| HDdb | 8 | 8 | 8 | 8 | 8 | 8 | 8 | 8 | 8 | 8 | 16 | 8 |
| QNds | 128 | 128 | 128 | 64 | 64 | 64 | 64 | 64 | 64 | 64 | 64 | 64 |
| 4PYdx | 128 | 128 | 128 | 128 | 128 | 128 | 128 | 128 | 128 | 128 | 128 | 128 |
| 4PYeh | 128 | 16 | 8 | 16 | 16 | 16 | 16 | 16 | 16 | 64 | 32 | 16 |

| | Reference | | | | | | | | | | | |
|---|---|---|---|---|---|---|---|---|---|---|---|---|
| | Iso- | ACTCC | NCTC | NCTC | | Iso- | Iso- | Iso- | Iso- | Iso- | NCTC | Iso- |
| | late 10 | 10541 | 7171 | 5957 | EBH1 | late 1 | late 2 | late 3 | late 4 | late 5 | 11047 | late 1 |
| | Strain | | | | | | | | | | | |
| | | | | E. fae- | E. fae- | E. fae- | E. fae- | E. fae- | E. fae- | E. fae- | S. epide- | S. epide- |
| Code | MRSA | E. faecium | E. faecium | calis | calis | calis | calis | calis | calis | calis | misis | misis |
| Fluclox. | 0.25 | 1 | 1 | 1 | 0.5 | 1 | 0.5 | — | 1 | 1 | 0.05 | >16 |
| Amp. | 8 | 2 | 1 | 2 | 0.05 | 0.05 | 0.05 | 0.05 | 2 | 2 | 4 | 8 |
| Vanc. | 0.12 | 0.12 | 0.12 | 0.25 | 0.12 | 0.25 | 0.25 | 0.12 | 16 | 2 | 8 | 8 |
| 3PYaf | 64 | 64 | 64 | 64 | 128 | 64 | 64 | — | 128 | 128 | 32 | 128 |
| 4PYaf | 64 | 64 | 64 | 64 | 64 | 64 | 64 | 64 | 128 | 128 | 64 | 64 |
| 4PYam | 32 | X | 256 | X | X | 256 | 256 | — | 256 | 256 | 256 | 128 |
| 3PYay | 256 | X | X | 128 | 128 | 128 | 128 | — | X | X | 128 | X |
| QNca | 128 | 256 | 256 | 256 | 256 | 256 | 256 | 256 | 256 | 256 | 64 | 64 |
| 2PYcb | X | X | 256 | X | X | X | X | X | X | X | X | 256 |
| PZcb | 256 | X | X | X | X | X | 256 | 256 | X | X | 256 | X |
| 3PYcc | 256 | X | 256 | 256 | X | 256 | 256 | — | 256 | 128 | 32 | X |
| QNcc | X | X | X | X | X | X | 256 | 256 | X | X | X | X |
| HDcd | 256 | 256 | 256 | 256 | 256 | 128 | 128 | 128 | 256 | 128 | 128 | 128 |
| cj | 4 | X | X | X | X | 256 | X | — | X | 256 | 2 | 2 |
| 2PYcj | 8 | X | X | X | X | X | X | 256 | X | 256 | 256 | 256 |
| 3PYcj | 8 | X | X | 256 | X | 128 | 128 | — | X | X | 64 | 128 |
| PZcj | 16 | X | X | X | X | X | X | 256 | X | X | X | 256 |
| HDcj | 64 | 128 | 128 | 128 | 128 | 128 | 128 | — | 64 | 128 | 64 | 64 |
| cl | X | X | X | X | X | X | X | X | X | X | X | X |
| 2PYcl | 64 | 128 | 256 | 256 | 256 | X | X | X | X | X | X | X |
| 3PYcl | 64 | 128 | 256 | 128 | 256 | 128 | 128 | — | 256 | 128 | 256 | 256 |
| QNcl | X | X | X | X | X | X | 256 | 256 | X | X | 256 | X |
| HDcl | 128 | 128 | 128 | 128 | 128 | 128 | 32 | 16 | 128 | 128 | 64 | 128 |
| 4PYcq | 4 | 4 | 4 | 4 | 4 | 4 | 4 | — | 4 | 4 | 4 | 4 |
| 4PYcr | X | X | X | X | X | X | X | X | X | X | X | X |
| 2PYdb | X | X | X | X | X | X | X | X | X | X | X | X |
| 3PYdb | 256 | X | X | X | X | X | 256 | — | X | 256 | 256 | 256 |
| 4PYdb | X | X | X | X | X | X | 256 | 256 | X | X | 128 | 128 |
| HDdb | 8 | 4 | 4 | 4 | 4 | 4 | 4 | 4 | 8 | 8 | 4 | 4 |
| QNds | 64 | 128 | 64 | 64 | 128 | 128 | 128 | 128 | 128 | 64 | 256 | 256 |
| 4PYdx | 128 | 256 | 256 | 256 | 256 | 256 | 256 | — | 256 | 256 | 64 | 128 |
| 4PYeh | 16 | X | X | X | X | X | X | X | X | X | 256 | 256 |

| | Reference | | | | | | | | | |
|---|---|---|---|---|---|---|---|---|---|---|
| | Iso- | | | | | NCTC | NCTC | NCTC | NCTC | NCTC |
| | late 1 | W3110R− | W3110R+ | 327 | 4444 | 6749 | 6017 | 10257 | 11582 | 8344 |
| | Strain | | | | | | | | | |
| | S. haemo- | | | K. pneu- | S. mar- | P. auri- | | S. malto- | | B. bronchi- |
| Code | lyticus | E. coli | E. coli | moniae | cesens | ginosa | S. aboni | philia | E. cloacae | septica |
| Fluclox. | 0.5 | >16 | >16 | >16 | >16 | 8 | 0.05 | 16 | >16 | >16 |
| Amp. | 1 | 1 | >16 | >16 | 8 | 16 | 0.05 | 16 | 16 | 1 |
| Vanc. | 8 | >16 | >16 | >16 | >16 | >16 | 0.05 | >16 | >16 | >16 |
| 3PYaf | 64 | 64 | 64 | X | 256 | 64 | — | 64 | X | X |
| 4PYaf | 64 | X | X | X | X | X | X | X | X | X |
| 4PYam | 256 | X | X | X | X | X | 32 | X | X | X |
| 3PYay | 256 | 256 | 256 | 256 | 256 | 128 | — | 256 | X | X |
| QNca | 128 | 256 | 256 | 256 | 256 | 256 | 256 | 256 | 32 | — |
| 2PYcb | X | X | X | X | X | X | X | X | X | 256 |
| PZcb | 256 | X | X | X | X | X | X | X | X | — |
| 3PYcc | X | X | X | X | X | X | 256 | X | X | X |
| QNcc | X | X | X | X | X | X | X | X | X | 256 |
| HDcd | 128 | 256 | 256 | 256 | 256 | 256 | 128 | 128 | 256 | 256 |
| cj | X | 256 | 256 | 256 | X | 256 | 1 | 256 | 256 | 256 |
| 2PYcj | 256 | X | X | X | X | X | X | X | X | — |
| 3PYcj | X | X | X | X | X | X | 256 | X | X | X |
| PZcj | X | X | X | X | X | X | X | X | X | — |
| HDcj | 128 | 128 | 128 | 128 | 256 | 256 | 128 | 128 | 256 | 128 |
| cl | X | X | X | X | X | X | X | X | X | X |
| 2PYcl | X | X | X | X | X | X | X | X | X | X |
| 3PYcl | 128 | X | X | X | X | X | 256 | X | X | X |
| QNcl | 256 | X | X | X | X | X | X | X | X | — |
| HDcl | 128 | 128 | 128 | 128 | 256 | 128 | 128 | 128 | 256 | 128 |
| 4PYcq | 4 | X | X | X | X | X | — | X | X | X |
| 4PYcr | X | X | X | X | X | X | — | X | X | X |
| 2PYdb | X | X | X | X | X | X | X | X | X | — |
| 3PYdb | 256 | 256 | 256 | 256 | X | 256 | 32 | 266 | 256 | 256 |
| 4PYdb | 128 | X | X | X | X | X | X | 128 | X | X |
| HDdb | 4 | X | X | X | X | X | X | X | X | X |
| QNds | 256 | X | X | X | X | X | X | 256 | X | — |
| 4PYdx | 256 | 256 | 128 | 256 | 256 | 32 | 32 | 128 | 256 | 256 |
| 4PYeh | 256 | X | X | X | X | X | X | X | X | X |

It will be noted that compound 4PYcq was active against all strains of MRSA as well as all other Gram positive bacteria. For this reason it was further tested against some vancomycin resistant enterococci, using the multipoint inoculator method described above. It can be seen from Table 5 that the high level activity was retained.

TABLE 5

| | Vancomycin | 4PYcq |
|---|---|---|
| Culture | MIC (μgml⁻¹) | MIC (μgml⁻¹) |
| 3001562 | 4-8 | 2-4 |
| 3002043 | 4-8 | |
| 3002066 | 4-8 | |
| 3005323 | >16 | |
| 3005426 | 4-8 | |
| 30053530 | >16 | |
| 3102095 | >16 | |

4Pycq was also tested against the following bacteria, using the multipoint inoculator method. Table 5a shows that 4Pycq did not inhibit the growth of a wide range of gram-negative bacteria.

TABLE 5a

| | 4PYcq |
|---|---|
| Microorganism | MIC (μgml⁻¹) |
| GRAM POSITIVE | |
| Bacillus cereus | 2-4 |
| Bacillus megarerium | 0.5-1 |
| Micrococcus roseus | 0.5-1 |
| Bacillus polymyxa | 2-4 |
| GRAM NEGATIVE | |
| Escherichia coli NCTC 10418 | — |
| Escherichia coli O157 NCTC 12900 | — |
| Escherichia coli ATCC 25922 | — |
| Pseudomonas aeruginosa ATCC 15692 | — |
| (PA01) | |
| Serratia marcescens | — |
| Enterobacter cloacae NCTC11582 | — |
| Enterobacter cloacae ATCC 13047 | — |
| Citrobacter diversus 2046E | — |
| Klebsiella pneumoniae ATCC 33495 | — |
| Proteus mirabilis NCTC 5887 | — |
| Salmonella pullorum | — |
| Salmonella arizonae | — |
| Salmonella banana | — |
| Salmonella malawi | — |
| Salmonella enteriditis | — |
| Salmonella Cambridge | — |
| Hafnia alvei | — |
| Citrobacter freundii | — |
| Shigella sonnei | — |
| Proteus mirabilis | — |
| Providencia alcalifaciens | — |

Toxicology

The in vitro assay used human mononuclear leucocytes (MNL, white blood cells) which were incubated with the test compound for 18 hours and cell death determined by tryptan blue exclusion (tryptan blue is a dye, which stains dead cells, whilst living cells extrude it). The main results of interest are shown in Table 6 below.

From Table 6, it can be seen that some direct MNL toxicity was encountered. The starting material pyridine-2-carboxamidrazone 2PY, aldehyde ae and the compound 102PYal were the most toxic compounds. However, the direct MNL toxicity for the other test compounds, 2PYae, 2PYaf, 2PYbe and 2PYeh, were indistinguishable from the background levels.

It is interesting to note that the starting material pyridine-2-carboxamidrazone 2PY was amongst the most cytotoxic compounds, but that generally, when it is combined with an aldehyde, even the very toxic aldehyde ae, the resulting benzylideneheteroarylcarboxamidrazone possesses no significant cytotoxicity. The exception to this is compound 2PYal, which is more cytotoxic than both of its starting materials.

TABLE 6
Results of direct leucocyte toxicity testing

| | | Cono. | % Leucocyte |
|---|---|---|---|
| Compound | Structure | (mM) | Death |
| Control- | — | — | 1.0 ± 1.1 |
| acetone- | | | 5.4 ± 1.1 |
| DMSO | | | |
| 2PY | 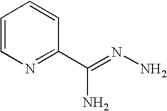 | 1.0 | 12.1 ± 2.2 |
| ae | 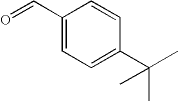 | 1.0 | 24.1 ± 9.6 |
| af | 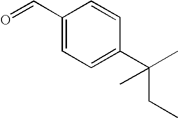 | 1.0 | 5.5 ± 3.0 |
| al | 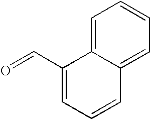 | 1.0 | 9.2 ± 4.7 |
| 2PYae | 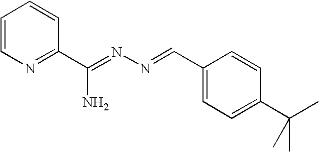 | 0.1 | 1.1 ± 1.0 |
| 2PYaf | 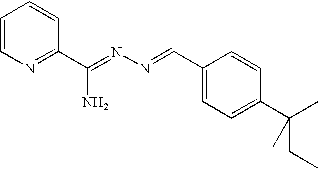 | 1.0 | 3.1 ± 1.0 |
| 2PYal | 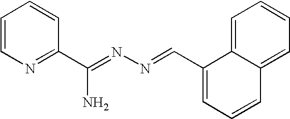 | 1.0 | 17.1 ± 4.9 |
| 2PYbe | 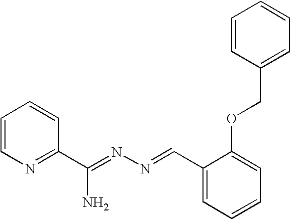 | 0.1 | 5.1 ± 4.1 |
| 2PYeh | 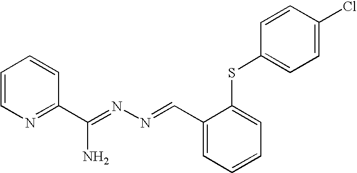 | 0.1 | 3.7 ± 2.6 |

It was investigated whether the amidrazones were oxidised to potentially reactive cytotoxic species. It is possible, for example, that the benzylideneheteroarylcarboxamidrazone could be cleaved, to produce the free pyridine-2-carboxamidrazone 2PY, which has already been shown to be toxic. To investigate the indirect toxicity of these compounds, Coleman et al (Preliminary in-vitro toxicological evaluation of a series of 2-pyridylcarboxamidrazone candidate anti-tuberculosis compounds. Environ. Tox. Pharmacol. 1999, 7, 59-65) repeated the toxicology experiments, but also added to these, rat liver microsomes as a metabolising system. It was discovered that generally, there was negligible bioactivation of the amidrazones to toxic species. The only compound which was affected by the metabolising system was 2PYal, which was also the only amidrazone to be directly toxic. For 2PYal there was actually a marked reduction in cytotoxicity in the presence of the metabolising system, suggesting that a biotransformationally mediated partial detoxification occurred.

Four out of the five tested benzylideneheteroarylcarboxamidrazones were not significantly toxic, indicating that in the rat, the amidrazone links are not cleaved by oxidative metabolism and that potentially cytotoxic substituents are not likely to be liberated in vivo. Studies with human microsomes would be necessary to confirm this.

The most promising antibacterial compound found in this study was N¹-[3,5-di-(tert-butyl)-2-hydroxy-benzylidene]-pyridine-2-carboxamidrazone 4PYcq. It was active against the MRSA strains and the vancomycin resistant enterococci which were tested, all with an MIC of 2-4 μgml⁻¹.

The toxicity of 4PYcq and the aldehyde cq and pyridine-4-carboxamidrazone precursors were assessed using the same in vitro assay with human mononuclear leucocytes as that described above. The toxicity of related compounds, 2-hydroxybenzylidene-pyridine-2-carboxamidrazone 4PYbw and benzylidenepyridine-2-carboxamidrazone 4PYaa were also examined.

Unfortunately, 4PYcq was found to be very toxic to leucocytes, causing lysis of the cells during the course of the experiment. Experiments on the two related compounds 4PYaa and 4PYbw show that these compounds are only mildly toxic in comparison, indicating that the t-butyl groups somehow afford huge cytotoxicity to 4PYcq. This may be due to the steric properties of the bulky t-butyl groups, or due to their lipophilicity. The toxicity of compound 4PYcq will preclude its use as a systemic anti-bacterial. However, it may still find utility in topical applications where systemic toxicity is less of an issue.

### EXPERIMENTAL

Pyridine-2-carboxamidrazone 2PY

Hydrazine hydrate (15 ml) was added to a solution of pyridine-2-carbonitrile (8.5 g, 81 mmol) in ethanol (15 ml) and left at RT for 2 days. The solution was then diluted with an equal volume of water, extracted with ethyl acetate [rather than with ether as in the method proposed by F. H. Case¹⁴⁵, as this gave poor yields] and dried over sodium sulfate. The solvent was then removed by rotary evaporation, at 30° C.-40° C., to give the product (8.17 g, 73%). R.f. [EtOAc:MeOH (2:1)]: 0.10. ¹H NMR: 5.33 (bs, 2H, NH₂), 5.73 (bs, 2H, NH₂), 7.31 (ddd, 1H, J=4.9, 2.4, 1.3 Hz, H4 or H5), 7.74 (ddd, 1H, J=6.9, 2.4, 1.8 Hz, H4 or H5), 7.90 (m, 1H, H3 or H6), 8.48 (m, 1H, H3 or H6). APCI-MS m/z: 137 (M+H)⁺.

Pyridine-3-carboxamidrazone 3PY

Hydrazine (80%, 21.5 ml) was added to a solution of pyridine-3-carbonitrile (6.4 g, 62 mmol) in ethanol (10 ml) and ether (10 ml). The mixture was left at RT, with stirring for five days, after which the majority of the solvent was removed by rotary evaporation, at 30° C.-40° C. The residual solution was cooled and a precipitate formed which was obtained by filtration and rapidly washed with ether to yield 6.00 g (55%). R.f. [EtOAc:MeOH (2:1)]: 0.10. ¹H NMR: 5.78 (bs, 4H, 2(NH₂)), 7.34 (m, 1H, H4 or H6), 7.99 (m, 1H, H4 or H6), 8.48 (dd, J=4.8, 1.6 Hz, H5), 8.86 (d, J=1.8 Hz, H2). APCI-MS m/z: 137 (M+H)⁺.

Pyridine-4-carboxamidrazone 4PY

Prepared from pyridine-4-carbonitrile, using the same method as pyridine-3-carboxamidrazone 3PY, to yield 5.00 g (67%). R.f. [EtOAc:MeOH (2:1)]: 0.18. ¹H NMR: 5.33 (bs, 2H, NH₂), 5.71 (bs, 2H, NH₂), 7.63 (d, 2H, J=6.3 Hz, Pyr-H), 8.50 (d, 2H, J=6.3 Hz, Pyr-H). APCI-MS m/z: 137 (M+H)⁺.

Pyrazine-2-carboxamidrazone PZ

Prepared from pyrazine-2-carbonitrile, using the same method as pyridine-2-carboxamidrazone 2PY to yield 8.61 g (66%). R.f. [EtOAc:MeOH (9:1)]: 0.23. ¹H NMR: 5.62 (bs, 2H, NH₂), 5.71 (bs, 2H, NH₂), 8.52 (s, 2H, H5 and H6), 9.10 (d, 1H, J=1.2 Hz, H3). APCI-MS m/z: 138 (M+H)⁺.

Quinoline-2-carboxamidrazone QN

Prepared from quinoline-2-carbonitrile, using the same method as pyridine-2-carboxamidrazone 2PY to yield 4.78 g (81%). R.f. [EtOAc:MeOH (9:1)]: 0.39. ¹H NMR: 5.66 (bs, 2H, NH₂), 5.92 (bs, 2H, NH₂), 7.55 (ddd, 1H, J=6.9 Hz, H6 or H7), 7.73 (ddd, 1H J=6.9 Hz, H6 or H7), 7.92 (d, 1H, J=8.4 Hz, H5 or H8), 8.00 (d, 1H, J=8.6 Hz, H5 or H8), 8.05 (d, 1H, J=8.8 Hz, H3 or H4), 8.23 (d, 1H, J=8.8 Hz, H3 or H4). APCI-MS m/z: 187 (M+H)⁺.

Automated Synthesis of the N¹-Benzylideneheteroarylcarboxamidrazone and Hydrazone Library

Glass 4 ml vials in a matrix were charged with 2-pyridylhydrazine HD (Aldrich) and each of the heteroarylcarboxamidrazones (0.4 mmol) in methanol (1 ml), with the exception 2-quinolylcarboxamidrazone, which was insoluble, so had to be weighed manually. This was followed by addition of an ethanolic solution of aldehyde (0.25M, 1.8 ml, 1.1 eq). The vials were heated in a heating block at 65° C. for one hour to remove the methanol, then at 75° C. for up to two hours, during which time the ethanol evaporated, to give the crude products. Purification was performed by robotic trituration (3×3 ml ether or petroleum ether depending upon the lipophilicity of the material). The products were then dried under high vacuum prior to analysis (yield range 60-97%).

All compounds were analysed by thin layer chromatography and positive APCI-MS (Table 7). Initially 10% of the compounds were analysed by ¹H NMR, then compounds which showed biological activity in the primary screen, were also analysed by ¹H NMR and purified, if necessary, usually by recrystalisation, prior to further biological testing. Certain compounds were also subjected to ¹³C NMR, infrared, melting point and elemental analyses.

TABLE 7
Analysis of the N¹-benzylideneheteroarylcarboxamidrazone library.

| | | | % | APCI-MS | | % |
|---|---|---|---|---|---|---|
| Compound | Appearance | MW | Yield | m/z | R.f. | Purity |
| 2PYab | Yellow crystals | 238 | 66 | 239 | 0.59 | 90 |
| 2PYac | Yellow solid | 252 | 56 | 253 | 0.53 | 98 |
| 2PYaf | Yellow solid | 294 | 80 | 295 | 0.63 | 98 |
| 2PYah | Yellow-brown solid | 238 | 74 | 239 | 0.55 | 98 |
| 2PYai | Yellow solid | 252 | 55 | 253 | 0.58 | |
| 2PYaj | Beige solid | 238 | 84 | 239 | 0.39 | |
| 2PYal | Yellow solid | 274 | 90 | 275 | 0.53 | 95 |
| 2PYar | Brown oil | 302 | 97 | 303 | 0.55 | 92 |
| 2PYat | Yellow solid | 268 | 57 | 269 | 0.47 | |
| 2PYau | Yellow solid | 282 | 82 | 283 | 0.51 | |
| 2PYav | Yellow crystals | 296 | 80 | 97 | 0.54 | 98 |
| 2PYaw | Yellow solid | 352 | 56 | 353 | 0.60 | |
| 2PYax | Brown solid | 254 | 92 | 255 | 0.53 | 98 |
| 2PYaz | Yellow solid | 282 | 38 | 283 | 0.54 | 80 |
| 2PYba | Yellow solid | 296 | 82 | 297 | 0.54 | |
| 2PYbb | Yellow solid | 310 | 41 | 311 | 0.55 | |
| 2PYbc | Yellow solid | 324 | 55 | 325 | 0.55 | |
| 2PYbd | Yellow solid | 254 | 73 | 255 | 0.22 | |
| 2PYbi | Yellow solid | 374 | 92 | 375 | 0.45 | |
| 2PYbj | Yellow solid | 388 | 77 | 389 | 0.46 | |
| 2PYbk | Yellow solid | 374 | 79 | 375 | 0.49 | |
| 2PYbl | Yellow solid | 416 | 83 | 417 | 0.49 | |
| 2PYbm | Yellow crystals | 405 | 43 | 406 | 0.53 | |
| 2PYbo | Yellow solid | 374 | 89 | 375 | 0.28 | 98 |
| 2PYbp | Yellow solid | 388 | 64 | 389 | 0.40 | 98 |
| 2PYbq | Yellow solid | 374 | 81 | 375 | 0.42 | 98 |
| 2PYbr | Yellow solid | 416 | 86 | 417 | 0.45 | |
| 2PYca | Brown solid | 256 | 73 | 257 | 0.30 | 72 |
| 2PYcb | Dark brown solid | 272 | 89 | 273 | 0.30 | 98 |
| 2PYcc | Brown solid | 272 | 66 | 273 | 0.25 | 68 |
| 2PYcf | Yellow crystals | 270 | 54 | 271 | 0.67 | 98 |
| 2PYcj | Brown solid | 285 | 82 | 286 | 0.30 | 98 |
| 2PYcl | Brown solid | 364 | 88 | 365, 367 | 0.47 | 65 |
| 2PYcm | Dark brown solid | 330 | 90 | 331 | 0.06 | 85 |
| 2PYcn | Brown solid | 364 | 75 | 365, 367 | 0.10 | |
| 2PYco | Yellow solid | 492 | 80 | 493 | 0.62 | |
| 2PYdb | Yellow solid | 290 | 48 | 291 | 0.69 | 98 |
| 2PYdc | Brown crystals | 304 | 67 | 305 | 0.73 | |
| 2PYdg | Yellow solid | 258 | 57 | 259, 261 | 0.60 | 98 |
| 2PYdm | Yellow solid | 292 | 86 | 293 | 0.66 | 98 |
| 2PYdu | Brown solid | 228 | 90 | 229 | 0.06 | |
| 2PYeb | Light brown solid | 292 | 76 | 105, 189, 293 | 0.85 | 98 |
| 2PYeh | Yellow solid | 366 | 75 | 367, 369 | 0.59 | 98 |
| 3PYac | Yellow solid | 252 | 89 | 253, 265* | 0.29 | |
| 3PYad | Yellow solid | 266 | 60 | 267, 293* | 0.30 | |
| 3PYaf | Orange solid | 294 | 59 | 295, 349* | 0.26, 0.84 | 98 |
| 3PYal | Orange solid | 274 | 76 | 275, 309* | 0.22, 0.78 | 67 |
| 3PYat | Yellow solid | 268 | 34 | 269, 296* | 0.20, 0.70 | |
| 3PYau | Yellow solid | 282 | 38 | 283, 324* | 0.20, 0.74 | |
| 3PYav | Yellow solid | 296 | 69 | 297, 353* | 0.21, 0.74 | 60 |
| 3PYaw | Yellow solid | 352 | 50 | 353 | 0.29, 0.80 | |
| 3PYax | Orange solid | 254 | 62 | 255, 269* | 0.28, 0.74 | |
| 3PYay | Yellow solid | 268 | 40 | 269, 297* | 0.05, 0.51 | 98 |
| 3PYaz | Yellow solid | 282 | 44 | 283, 325* | 0.36, 0.74 | |
| 3PYba | Yellow solid | 296 | 70 | 297, 353* | 0.39, 0.72 | 98 |
| 3PYbb | Yellow solid | 310 | 37 | 311, 381* | 0.43, 0.75 | |
| 3PYbc | Yellow solid | 324 | 46 | 325, 409* | 0.44, 0.76 | |
| 3PYbm | Yellow solid | 405 | 46 | 406 | 0.17 | |
| 3PYcb | Brown solid | 272 | 92 | 274, 305* | 0.16 | |
| 3PYcc | Brown solid | 272 | 79 | 273, 305* | ^{a}0.45 | 60 |
| 3PYcj | Yellow solid | 285 | 83 | 285, 331* | ^{a}0.52, 0.80 | 60 |
| 3PYcl | Orange solid | 364 | 82 | 365, 367 | ^{a}0.49 | 63 |
| 3PYdb | Brown solid | 290 | 92 | 291, 341* | 0.30 | 67 |
| 4PYac | Yellow solid | 252 | 38 | 253 | 0.56 | |
| 4PYad | Yellow solid | 266 | 86 | 267 | 0.40 | |
| 4PYaf | Yellow solid | 294 | 60 | 295 | 0.38 | 98 |
| 4PYam | Orange crystals | 324 | 87 | 325 | 0.32 | 98 |
| 4PYao | Yellow solid | 250 | 64 | 251 | 0.35 | 90 |
| 4PYar | Brown solid | 302 | 42 | 303 | 0.48 | |
| 4PYat | Yellow solid | 268 | 36 | 269 | 0.56 | |
| 4PYau | Yellow solid | 282 | 75 | 283 | 0.62 | |
| 4PYav | Yellow solid | 296 | 54 | 297 | 0.67 | 98 |
| 4PYaw | Yellow solid | 352 | 85 | 353 | 0.70 | |
| 4PYax | Yellow solid | 254 | 83 | 255 | 0.17 | 98 |
| 4PYaz | Yellow solid | 282 | 36 | 283 | 0.38 | |
| 4PYba | Yellow solid | 296 | 48 | 297 | 0.39 | 88 |
| 4PYbb | Yellow solid | 310 | 68 | 311 | 0.40 | |
| 4PYbc | Yellow solid | 324 | 42 | 325 | 0.42 | |
| 4PYbi | Yellow solid | 374 | 88 | 375 | 0.20 | 92 |
| 4PYbj | Yellow solid | 388 | 77 | 389 | 0.20 | 98 |
| 4PYbk | Yellow solid | 374 | 82 | 375 | 0.18 | |
| 4PYbl | Yellow sold | 416 | 87 | 417 | 0.20 | |
| 4PYbm | Yellow solid | 405 | 58 | 406 | 0.19 | |
| 4PYbo | Yellow solid | 374 | 78 | 375 | 0.25 | 98 |
| 4PYbp | Yellow solid | 388 | 56 | 389 | 0.29 | 98 |
| 4PYbq | Yellow solid | 374 | 68 | 375 | 0.23 | |
| 4PYbr | Yellow solid | 416 | 71 | 417 | 0.27 | |
| 4PYcb | Brown solid | 272 | 99 | 273 | 0.25 | |
| 4PYcc | Red solid | 272 | 96 | 273 | ^{a}0.40 | |
| 4PYcj | Yellow solid | 285 | 86 | 286 | ^{a}0.49 | |
| 4PYcl | Yellow solid | 364 | 91 | 365, 367 | ^{a}0.48 | |
| 4PYcm | Yellow solid | 330 | 85 | 331 | ^{a}0.45 | |
| 4PYcn | Orange solid | 364 | 92 | 365, 367 | 0.02 | 98 |
| 4PYco | Yellow solid | 492 | 78 | 493 | 0.33 | |
| 4PYcq | Yellow crystals | 352 | 60 | 353 | 0.48 | 98 |
| 4PYcr | Yellow solid | 352 | 57 | 353 | 0.48 | 74 |
| 4PYcs | Orange solid | 284 | 45 | 285 | 0.34 | |
| 4PYct | Orange solid | 284 | 62 | 285 | 0.37 | |
| 4PYdb | Brown solid | 290 | 55 | 291 | 0.41 | 98 |
| 4PYdc | Yellow solid | 304 | 48 | 305 | 0.30 | 75 |
| 4PYdm | Yellow solid | 292 | 81 | 293 | 0.49 | |
| 4PYdu | Brown solid | 228 | 89 | 229 | 0.06 | |
| 4PYdx | Orange solid | 317 | 70 | 200, 318 | 0.20 | 83 |
| 4PYeb | Dark orange solid | 292 | 65 | 293 | 0.70 | |
| 4PYec | Orange-brown solid | 306 | 61 | 105 203, 307 | 0.52 | |
| 4PYeh | Yellow solid | 366 | 56 | 367, 369 | 0.49 | 98 |
| PZar | Orange solid | 303 | 64 | 304 | 0.64 | |
| PZaw | Yellow solid | 353 | 74 | 354 | 0.65 | 98 |
| PZax | Yellow solid | 255 | 97 | 256 | 0.61 | |
| PZaz | Yellow solid | 283 | 51 | 284 | 0.60 | |
| PZba | Yellow solid | 297 | 66 | 298 | 0.61 | |
| PZbb | Yellow solid | 311 | 39 | 312 | 0.62 | |
| PZbc | Yellow solid | 325 | 46 | 326 | 0.63 | |
| PZbm | Yellow solid | 406 | 83 | 407 | 0.62 | |
| PZca | Orange solid | 257 | 54 | 258 | 0.48 | 98 |
| PZcb | Yellow solid | 273 | 70 | 274 | 0.39 | 98 |
| PZcc | Yellow-brown solid | 273 | 50 | 274 | 0.40 | 98 |
| PZcj | Yellow solid | 286 | 93 | 287 | 0.41 | 98 |
| PZdu | Yellow solid | 229 | 72 | 230 | 0.08 | |
| QNar | Orange solid | 352 | 61 | 353 | 0.79 | 85 |
| QNaw | Yellow solid | 402 | 53 | 403 | 0.81 | |
| QNax | Yellow solid | 304 | 86 | 305 | 0.74 | |
| QNaz | Yellow solid | 332 | 39 | 333 | 0.76 | 90 |
| QNba | Yellow solid | 346 | 53 | 347 | 0.78 | |
| QNbb | Yellow solid | 360 | 48 | 361 | 0.77 | |
| QNbc | Yellow solid | 374 | 60 | 375 | 0.78 | |
| QNbm | Yellow solid | 455 | 72 | 456 | 0.80 | 75 |
| QNca | Yellow solid | 306 | 41 | 307 | 0.54 | 98 |
| QNcb | Yellow solid | 322 | 63 | 155, 323 | 0.50 | |
| QNcc | Brown solid | 322 | 68 | 323 | 0.47 | 98 |
| QNds | Yellow solid | 375 | 77 | 376 | 0.38 | 98 |
| QNdy | Yellow solid | 313 | 81 | 314 | 0.83 | |
| HDac | Light orange solid | 225 | 66 | 226 | 0.77 | |
| HDad | Light orange solid | 239 | 70 | 240 | 0.75 | |
| HDaw | Light orange solid | 325 | 80 | 326 | 0.61 | |
| HDax | Light orange | 227 | 54 | 228 | 0.42 | 98 |
| | crystals | | | | | |
| HDaz | Light orange solid | 255 | 66 | 256 | 0.42 | |
| HDba | Light orange solid | 269 | 81 | 270 | 0.41 | |
| HDbb | Light orange solid | 283 | 61 | 284 | 0.43 | |
| HDbc | Light orange solid | 297 | 52 | 298 | 0.44 | |
| HDbm | Yellow-orange solid | 378 | 53 | 379 | 0.62 | |
| HDbo | Off white solid | 347 | 68 | 348 | 0.47 | |
| HDbq | White solid | 347 | 66 | 348 | 0.49 | 85 |
| HDbz | Red-brown solid | 229 | 56 | 230 | 0.22 | |
| HDcb | Red-brown solid | 245 | 59 | 246 | 0.26 | 98 |
| HDcc | Brown crystals | 245 | 95 | 246 | ^{a}0.43 | 80 |
| HDcd | Orange-brown solid | 245 | 83 | 246 | ^{a}0.50 | 98 |
| HDce | Brown solid | 243 | 82 | 244 | 0.60 | |
| HDcf | Brown solid | 243 | 45 | 244 | 0.65 | |
| HDcj | Yellow solid | 258 | 66 | 259 | ^{a}0.54 | 98 |
| HDcl | Orange solid | 337 | 76 | 338, 340 | ^{a}0.53 | 98 |
| HDdb | Brown solid | 263 | 71 | 264 | 0.66 | 98 |
| HDdm | Light orange solid | 265 | 74 | 266 | 0.73 | |
| HDdn | Light orange solid | 242 | 80 | 243 | 0.64 | |

In most cases R. f. values were determined using ethyl acetate as the eluent.
^{a}denotes that an ethyl acetate/methanol (9:1) mixture was used as the TLC eluent. Where two R. f. or m/z values are given, the underlined value was the most prominent.
*represents the m/z value for the 3PY reaction bis-substituted by-product. % Yield refers to the crude product yield. Purity, where given, has been estimated by ¹H NMR.

Full Characterisation of Selected Derivatives

### 3PYaf N¹-[4-(1,1-dimethylpropyl)benzylidene]-pyridine-3-carboxamidrazone

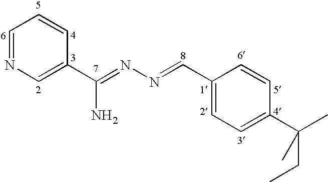

Recrystalised from ethanol three times, to give a yellow crystalline solid, 49% yield. R.f. [EtOAc]: 0.26. ¹H NMR (D₆DMSO): 0.63 (t, 3H, J=7.4 Hz, CH₂CH₃), 1.26 (s, 6H, CMe₂), 1.63 (q, 2H, J=7.4 Hz, CH₂CH₃), 7.12 (bs, 2H, NH₂), 7.38 (d, 2H, J=8.3 Hz, 3′H and 5′H), 7.47 (m, 1H, Pyr-H4), 7.82 (d, 2H, J=8.3 Hz, 2′H and 6′H), 8.26 (m, 1H, Pyr-H5), 8.42 (s, 1H, ═CHAr), 8.65 (m, 1H, Pyr-H6), 9.10 (m, 1H, Pyr-H2) ppm. ¹³C NMR (D₆CDCl₃): 9.0 (CH₂CH₃), 28.2 (CMe₂), 36.6 (CMe₂), 38.1 (CH₂CH₃), 123.3 (C5), 126.2 (C3′ and C5′), 127.6 (C2′ and C6′), 129.8 (C4), 132.0 (C1′), 134.2 (C6), 147.7 (C2), 151.3 (C3), 152.2 (C4′), 156.6 (C8), 156.7 (C8) ppm. IR (KBr disc): 3446 (νₐₛ NH₂), 3286 (νₛ NH₂), 3110 (ν Ar—CH), 3035 (ν Ar or Pyr-CH), 2963 (νₐₛ Me), 2869 (νₛ Me), 1622 (ν C═N), 1590 (ν skeletal Ar or Pyr), 1551 (ν skeletal Pyr), 1525 (ν skeletal Ar or Pyr), 1450 (δₐₛ Me or ν skeletal Ar or Pyr), 1378 (δₛ Me), 1332, 1303, 1193, 1106 (ν C—N), 1016, 960, 839 (γ CH, p-subst. Ar), 817 (γ CH, 3-Pyr), 711 (β ring, 3-Pyr), 630 cm⁻¹. APCI-MS m/z: 295(M+H)⁺. mp (corrected): 160.5-161.3° C. CHN Analysis, % m/m (% calculated/% found): C, 73.44/73.56; H, 7.53/7.53; N, 19.03/19.06.

### 4PYaf N¹-[4-(1,1-dimethylpropyl)benzylidene]-pyridine-4-carboxamidrazone

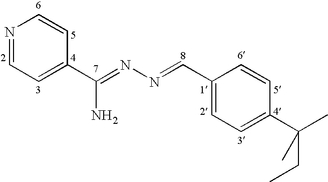

Recrystalised from toluene twice, to give a yellow crystalline solid, 42% yield. R.f. [EtOAc]: 0.38. ¹H NMR (D₆DMSO): 0.63 (t, 3H, J=7.5 Hz, CH₂CH₃), 1.26 (s, 6H, CMe₂), 1.64 (q, 2H, J=7.5 Hz, CH₂CH₃), 7.14 (bs, 2H, NH₂), 7.39 (d, 2H, J=8.3 Hz, 3′H and 5′H), 7.83 (d, 2H, J=8.3 Hz, 2′H and 6′H), 7.88 (dd, 2H, J=4.6, 1.6 Hz, Pyr-H3 and H5), 8.44 (s, 1H, ═CHAr), 8.67 (dd, 2H, J=4.6, 1.6 Hz, Pyr-H2 and H6) ppm. ¹³C NMR (D₆CDCl₃): 9.1 (CH₂CH₃), 28.3 (CMe₂), 36.7 (CMe₂), 38.1 (CH₂CH₃), 120.6 (C3 and C5), 126.3 (C2′ and C6′), 127.8 (C3′ and C5′), 131.9 (C1′), 141.3 (C4), 150.2 (C2 and C6), 152.5 (C4′), 156.6 (C7), 157.3 (C8) ppm. IR (KBr disc): 3443 (νₐₛ NH₂), 3274 (νₛ NH₂), 3122 (ν Ar—CH), 3019 (ν Ar or Pyr-CH), 2962 (νₐₛ Me), 2860 (νₛ Me), 1625 (ν C═N), 1595 (ν skeletal Ar or Pyr), 1568 (ν skeletal Pyr), 1521 (ν skeletal Ar or Pyr), 1449 (δₐₛ Me or ν skeletal Ar or Pyr), 1378 (δₛ Me), 1344, 1307, 1230, 1178, 1117, 1086 (ν C—N), 996, 878, 832 (γ CH, p-subst. Ar), 802 (γ CH, 4-Pyr), 748 (β ring, 4-Pyr), 701, 675 cm⁻¹. APCI-MS m/z: 295 (M+H)⁺. mp (corrected): 144.0-145.8° C. CHN Analysis, % m/m (% calculated/% found): C, 73.44/73.51; H, 7.53/7.41; N, 19.03/19.14.

### 4PYdx N¹-[(4-dimethylamino)-1-naphthylidene]-pyridine-4-carboxamidrazone

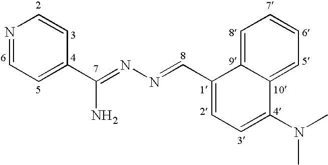

Orange oil, obtained by trituration with 60-80 petroleum ether, 86% yield. R.f. [EtOAc]: O.20. ¹H NMR (D₆DMSO): 2.90 (s, 6H, NMe₂), 7.11 (bs, 2H, NH₂), 7.15 (d, 1H, J=8.0 Hz, 2′ or 3′H), 7.54-7.66 (ov.m, 2H, 2Ar—H), 7.93 (dd, 2H, J=4.5, 1.5 Hz, Pyr-H3 and H5), 8.12 (d, 1H, J=8.0 Hz, 2′ or 3′H), 8.22 (m, 1H, Ar—H), 8.70 dd, 2H, J=4.5, 1.5 Hz, Pyr-H2 and H6), 8.94 (m, 1H, Ar—H), 9.07 (s, 1H, ═CHAr) ppm. ¹³C NMR (D₆CDCl₃): 44.8 (NMe₂), 113.0 (C3′), 120.6 (C3 and C5), 124.4 (C5′ or C6′ or C7′ or C8′), 124.7 (C5′ or C6′ or C7′ or C8′), 124.9 (C5′ or C6′ or C7′ or C8′), 125.0 (C5′ or C6′ or C7′ or C8′), 126.9 (C2′), 128.3 (C10′), 129.3 (C1′ or C9′), 132.6 (C1′ or C9′), 141.5 (C4), 150.1 (C2 and C6), 153.6 (C4′), 156.1 (C7), 157.2 (C8) ppm. IR (CHCl₃): 3505 (νₐₛ NH₂), 3388 (νₛ NH₂), 3006 (ν Ar or Pyr-CH), 2968 (ν sat. CH), 1619 (ν C═N), 1599 (ν skeletal Ar or Pyr), 1556 (ν skeletal Pyr), 1539, 1454 (ν skeletal Ar or Pyr)cm⁻¹. APCI-MS m/z: 317 (M+H)⁺. CHN Analysis, % m/m (% calculated/% found): C, 71.90/69.97; H, 6.03/6.06; N, 22.06/22.21.

### 4PYeh N¹-[2-(4-chlorothiophenyl)benzylidene]-pyridine-4-carboxamidrazone

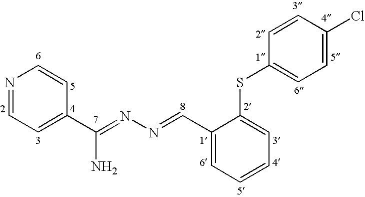

Recrystalised from ethanol three times, to give a yellow crystalline solid, 30% yield. R.f. [EtOAc]: 0.49. ¹H NMR (D₆DMSO): 7.22-7.37 (ov.m, 5H, NH₂ and 3″H and 5″H and Ar—H), 7.41-7.48 (ov.m, 4H, 2″H and 6″H and 2Ar—H), 7.86 (dd, 2H, J=4.5, 1.6 Hz, Pyr-H3 and H5), 8.35 (m, 1H, Ar—H), 8.67 (dd, 2H, J=4.5, 1.6 Hz, Pyr-H2 and H6), 8.81 (s, 1H, ═CHAr) ppm. ¹³C NMR (D₆CDCl₃): 120.7 (C3 and C5), 127.0 (C5′), 129.6 (C2″ and C6″), 129.7 (C3′), 130.4 (C6′), 131.5 (C4′), 133.0 (C3″ and C5″), 133.5 (C1′ or C1″ or C4″), 133.7 (C1″ or C1″ or C4″), 133.9 (C1′ or C1″ or C4″), 136.4 (C2″), 141.1 (C4), 150.3 (C2 and C6), 155.7 (C8), 157.3 (C7) ppm. IR (KBr disc): 3409 (νₐₛ NH₂), 3295 (νₛ NH₂), 3089 (ν Ar—CH), 3059 (ν Ar or Pyr-CH), 2972, 1610 (ν C═N), 1533 (ν skeletal Pyr), 1473 (ν skeletal Ar or Pyr), 1436 (ν skeletal Ar or Pyr), 1410, 1340, 1284, 1209, 1095 (ν CN), 1016, 999, 819 (γ CH, 4-Pyr or γ CH, p-subst. Ar), 766 (γ CH, o-subst. Ar), 741 cc, 667 cm⁻¹. APCI-MS m/z: 367, 369 (M+H)⁺. mp (corrected): 154.8-156.0° C. CHN Analysis, % m/m (% calculated/% found): C, 62.20/61.59; H, 4.12/3.87; N, 15.27/14.91.

### 4PYam N¹-(9-anthrylidene)-pyridine-2-carboxamidrazone

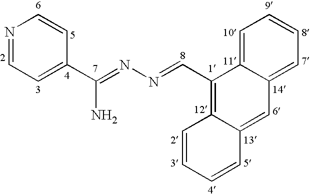

Recrystalised from methanol twice, to give an orange crystalline solid, 53% yield. R.f. [EtOAc]: 0.32. ¹H NMR (D₆DMSO): 7.17 (bs, 2H, NH₂), 7.61 (m, 4H, 4Ar—H), 8.00 (dd, 2H, J=4.5 Hz, 1.6 Hz, Pyr-H3 and H5), 8.16 (m, 2H, 2Ar—H), 8.71 (ov.m, 5H, Pyr-H2 and H6 and 3Ar—H), 9.66 (s, 1H, ═CHAr) ppm. ¹³C NMR (D₆CDCl₃): 120.7 (C3 and C5), 125.3 (C2′ and C10′ or C3′ and C9′ or C4′ and C8′ or C5′ and C7′), 125.4 (C2′ and C10′ or C3′ and C9′ or C4′ and C8′ or C5′ and C7′), 126.6 (C1′), 126.8 (C2′ and C10′ or C3′ and C9′ or C4′ and C8′ or C5′ and C7′), 128.9 (C2′ and C10′ or C3′ and C9′ or C4′ and C8′ or C5′ and C7′), 129.7 (C6′ or C11′ and C12′ or C13′ and C14′), 130.4 7 (C6′ or C11′ and C12′ or C13′ and C14′), 131.4 7 (C6′ or C11′ and C12′ or C13′ and C14′), 141.3 (C4), 150.5 (C2 and C6), 156.4 (C8), 157.3 (C7) ppm. IR (KBr disc): 3427 (νₐₛ NH₂), 3305 (νₛ NH₂), 3106 (ν Ar—CH), 3037 (ν Ar or Pyr-CH), 1621 (ν C═N), 1594 (ν skeletal Ar or Pyr), 1511 (ν skeletal Ar or Pyr), 1415 (ν skeletal Ar or Pyr), 1133 (ν C—N), 997, 889, 820 (γ CH, 4-Pyr), 734 (β ring, 4-Pyr), 674 cm⁻¹. APCI-MS m/z: 325 (M+H)⁺. mp (corrected): 241.9-244.1° C. CHN Analysis, % m/m (% calculated/% found): C, 77.76/77.46; H, 4.97/5.02; N, 17.27/6.93.

### 4PYcq N¹-[3,5-di-(tert-butyl)-2-hydroxybenzylidene]-pyridine-2-carboxamidrazone

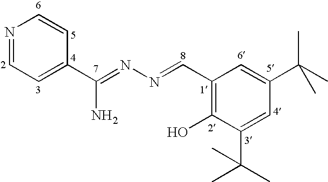

Recrystalised from methanol/40-60 PE to give a yellow solid, 68% yield. R.f. [EtOAc]: 0.48. ¹H NMR (D₆DMSO): 1.27 (s, 9H, CMe₃), 1.42 (s, 9H, CMe₃), 7.18 (bs, 2H, NH₂), 7.30 (d, 1H, J=2.4 Hz, 4′H), 7.34 (d, 1H, J=2.3 Hz, 6′H), 7.87 (d, 2H, J=6.1 Hz, Pyr-H3 and H5), 8.67-8.69 (ov.m, 3H, ═CHAr and Pyr-H2 and H6), 11.60 (bs, 1H, OH) ppm. ¹³C NMR (D₆CDCl₃): 29.4 (CMe₃), 31.4 (CMe₃), 34.1 (CMe₃), 35.0 (CMe₃), 117.4 (C1′), 120.6 (C3 and C5), 126.5 (C4′), 127.2 (C6′), 136.3 (C3′), 141.2 (C4), 150.3 (C2 and C6), 154.3 (C5′), 156.1 (C2′), 162.4 (C8) ppm. IR (KBr disc): 3468 (νₐₛ NH₂), 3282 (νₛ NH₂), 3250-3000 (ν OH, overlapping ν Ar—CH), 2954 (ν sat. CH), 2865 (ν sat. CH), 1633 (ν C═N), 1610 (ν skeletal Ar or Pyr), 1595 (ν skeletal Ar or Pyr), 1534 (ν skeletal Pyr), 1463 (ν skeletal Ar or Pyr), 1436 (ν skeletal Ar or Pyr), 1374, 1247, 1178, 1070 (ν C—N), 997, 968, 877, 818 (γ CH, 4-Pyr), 746 (β ring, 4-Pyr), 713, 642 cm⁻¹. APCI-MS m/z: 353 (M+H)⁺. mp corrected: 156.7-158.0° C. CHN Analysis, % m/m (% calculated/% found): C, 71.56/71.70; H, 8.01/7.96; N, 15.89/16.01.

### EXAMPLE 2

### Synthesis of N¹-benzylideneheteroarylcarboxamidrazones-N-oxides

A smaller variety of aldehydes were investigated and are referred to throughout by a two lower-case letter code. The structure of the aldehyde residues are shown in Table 2 above, and are referenced by the same two letter code for convenience. The aldehydes used are all commercially available.

The heterocarboxamidrazone-N-oxide building block 4PYO was prepared by the action of hydrazine monohydrate upon the corresponding cyano compound, which method was also used to synthesise the corresponding 3PYO building block.

General Method for the Preparation of N¹-Arylidene-pyridine-4-carboxamidrazone-N-oxides and N¹-Arylidene-pyridine-3-carboxamidrazone-N-oxides

A mixture of the pyridine carboxamidrazone N-oxide and an appropriate aldehyde (1.1-1.3 molar equivalents) in ethanol (20 mL/g of carboxamidrazone N-oxide) was stirred at reflux for 18 hours. The starting materials dissolved once heating commenced. After cooling, the precipitated material was collected by filtration, washed with a little cold ethanol and dried under vacuum. The material obtained at this point was generally found to contain a single component as judged by thin layer chromatography. If necessary the material was purified by recrystallisation.

Anti-Bacterial Activity

Table 8 shows the results of testing compound 4PYOcq against a range of gram positive and gram negative bacteria. The compound was tested as follows. Culture media (described previously) were prepared containing concentrations of the test compounds ranging from 128-0.0625 μg/mL using a doubling dilution method and placed (100 μL aliquots) in the wells of a transparent 96-well microtitre plate. The wells were inoculated with organism (50 μμL of medium containing 10⁶ cfu/mL) and the plates were incubated at 37° C. overnight. Where growth of organism occurred this was observed as a small button when viewed from underneath the plate. The MIC was determined as the lowest concentration inhibiting growth.

TABLE 8

| Microorganism | MIC (μg/ml) |
|---|---|
| GRAM POSITIVE | |
| Staphylococcus epidermidis NCTC 11047 | 32-64 |
| Enterococcus faecium ATCC 10541 | 32-64 |
| Bacillus cereus | 16-32 |
| Bacillus megarerium | 32-64 |
| Bacillus subtilis | 64-128 |
| Streptococcus bovis NCTC 11436 | 64-128 |
| Enterococcus faecalis NCTC 5957 | 64-128 |
| Enterococcus faecium NCTC 7171 | 32-64 |
| Staphylococcus aureus (MRSA) 96-7475 | 64-128 |
| Staphylococcus aureus NCTC 6571 | 16-32 |
| Micrococcus luteus | 8-16 |
| Micrococcus roseus | 1-2 |
| Bacillus polymyxa | 1-2 |
| GRAM NEGATIVE | |
| Moraxella catarrhalis | 32-64 |
| Escherichia coli olli-blue | — |
| Pseudomonas aeruginosa ATCC 15692 (PA01) | — |
| Serratia marcescens 4444 | — |
| Enterobacter cloacae ATCC 13047 | — |
| Citrobacter diversus 2046E | — |
| Klebsiella pneumoniae ATCC 33495 | — |
| Proteus mirabilis NCTC 5887 | — |
| Salmonella pullorum | — |
| Salmonella arizonae | — |
| Salmonella enteriditis | — |
| Hafnia alvei | — |
| Citrobacter freundii | — |
| Shigella sonnei | — |

Table 9a shows the results of testing the other compounds made against a range of gram positive and Table 9b against a range of gram negative bacteria, where the results are obtained as above and are given as the MIC (μg/ml).‘-’ indicates no inhibition of growth.

TABLE 9a

| | Compound - 4PYOxx | | | | | | | | | | |
|---|---|---|---|---|---|---|---|---|---|---|---|
| Microorganism | al | am | eh | fa | fb | fc | fd | fe | ff | fg | fi |
| Staphylococcus aureus W11 MRSA | — | — | — | — | — | — | 64-128 | — | 32-64 | — | 4-8 |
| Staphylococcus epidermidis NCTC | | | | | | | | | 32-64 | | 2-4 |
| 11047 | | | | | | | | | | | |
| Enterococcus faecium ATCC 10541 | 16-32 | 64-128 | — | 16-32 | 16-32 | 16-32 | 64-128 | — | 32-64 | — | 8-16 |
| Bacillus cereus | 8-16 | — | — | 8-16 | 16-32 | 16-32 | 64-128 | — | 32-64 | — | 8-16 |
| Bacillus megarerium | 16-32 | — | — | 16-32 | 16-32 | 16-32 | — | — | — | — | 8-16 |
| Bacillus subtilis | 16-32 | 64-128 | — | 4-8 | 8-16 | 31-64 | 64-128 | — | 32-64 | — | 8-16 |
| Staphylococcus aureus NCTC 10788 | — | — | — | — | — | 16-32 | — | — | 32-64 | — | 4-8 |
| Streptococcus bovis NCTC 11436 | — | — | — | — | — | 32-64 | 32-64 | — | 32-64 | 32-64 | 4-8 |
| Enterococcus faecalis NCTC 5957 | — | 64-128 | — | — | — | — | 64-128 | — | 32-64 | — | 4-8 |
| Staphylococcus aureus (MRSA) | | | | | | | | | 32-64 | | 4-8 |
| 96-7475 | | | | | | | | | | | |
| Staphylococcus aureus NCTC 6571 | — | — | — | — | — | — | — | — | 32-64 | — | 4-8 |
| Micrococcus luteus | 16-32 | — | — | — | — | 8-16 | 64-128 | 8-16 | 32-64 | 8-16 | 8-16 |
| Micrococcus roseus | 16-32 | 32-64 | 32-64 | 16-32 | 8-16 | 16-32 | 64-128 | — | — | — | 8-16 |

TABLE 9b

| | Compound - 4PYOxx | | | | | | | | | | |
|---|---|---|---|---|---|---|---|---|---|---|---|
| Microorganism | al | am | eh | fa | fb | fc | fd | fe | ff | fg | fi |
| Moraxella catarrhalis | 16-32 | — | 64-128 | — | 32-64 | 8-16 | 32-64 | 8-16 | 16-32 | 4-8 | 16-32 |
| Escherichia coli olli-blue | — | — | — | — | — | — | — | — | — | — | 16-32 |
| Pseudomonas aeruginosa ATCC | — | — | — | — | — | — | — | — | — | — | 32-64 |
| 15692 (PA01) | | | | | | | | | | | |
| Enterobacter cloacae ATCC 13047 | — | — | — | — | — | — | — | — | — | — | 32-64 |
| Citrobacter diversus 2046E | — | — | — | — | — | — | — | — | — | — | 32-64 |
| Klebsiella pneumoniae ATCC 33495 | — | — | — | — | — | — | — | — | — | — | 32-64 |
| Proteus mirabilis NCTC 5887 | — | — | — | — | — | — | — | — | — | — | 32-64 |
| Salmonella pullorum | — | — | — | — | — | — | — | — | — | — | — |
| Salmonella arizonae | — | — | — | — | — | — | — | — | — | — | 16-32 |
| Salmonella banana | — | — | — | — | — | — | — | — | — | — | 4-8 |
| Salmonella malawi | — | — | — | — | — | — | — | — | — | — | — |
| Salmonella enteriditis | — | — | — | — | — | — | — | — | — | — | 32-64 |
| Salmonella Cambridge | — | — | — | — | — | — | — | — | — | — | 16-32 |
| Hafnia alvei | — | — | — | — | — | — | — | — | — | — | 32-64 |
| Citrobacter freundii | — | — | — | — | — | — | — | — | — | — | 32-64 |
| Shigella sonnei | — | — | — | — | — | — | — | | | | |

### EXPERIMENTAL

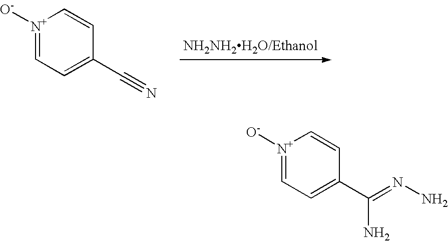

1-Oxy-isonicotinonitrile (14.516 g, 0.121 mol) was suspended in ethanol (45 mL) and treated with hydrazine monohydrate (30 mL) and stirred at ambient temperature for nine days. The solid material was collected by filtration, washed with ethanol (3×30 mL) and dried under vacuum to give the product as a yellow crystalline solid. Yield 15.46 g, 0.102 mol, 84%; MS (APCI +ve) m/z=153 (M+H)⁺; ¹H NMR (D6-DMSO; δ DMSO=2.50 ppm) 5.32 (bs, 2H, NH₂), 5.71 (bs, 2H, NH₂), 7.64 (d, 2H, J=7.2 Hz, Ar 2-H and Ar 6-H), 8.13 (d, 2H, J=7.2 Hz, Ar 3-H and Ar 5-H) ppm.

4PYOcq

Yellow crystalline solid. 68% yield; MP 277.2-278.9° C.; MS (APCI +ve) m/z=369 (M+H)⁺; IR (KBr disc) ν=3458, 3273, 3114, 2955, 1635, 1622, 1542, 1500, 1438, 1399, 1356, 1247 (N—O), 1177 cm⁻¹; ¹H NMR (D6-DMSO; δDMSO=2.50 ppm) 1.28 (s, 9H, C(CH₃)₃), 1.43 (s, 9H, C(CH₃)₃), 7.12 (bs, 2H, NH₂), 7.30 (d, 1H, J=2.4 Hz, Ar′—H), 7.33 (d, 1H, J=2.4 Hz, Ar′—H), 7.94 (d, 2H, J=7.3 Hz, Ar 2-H and Ar 6-H), 8.30 (d, 2H, J=7.3 Hz, Ar 3-H and Ar 5-H), 11.57 (s, 1H, N═CH) ppm

4PYOal

Yellow crystalline solid. 78% yield; MP 225.3-227.7° C.; MS (APCI +ve) m/z=291 (M+H)⁺, 273 (M−H₂O)⁺; IR (KBr disc) ν=3406, 3220, 3094, 1615, 1492, 1436, 1241 (N—O), 1181 cm⁻¹; ¹H NMR (D6-DMSO; δDMSO=2.50 ppm) 7.22 (bs, 2H NH₂), 7.55-7.70 (overlapping m, 3H, ArH), 7.98-8.04 (overlapping m, 4H, ArH), 8.27-8.34 (overlapping m, 3H, ArH), 8.81 (d, 1H, J=8.3 Hz, ArH), 9.19 (s, 1H, N═CH) ppm

4PYOfa

Yellow crystalline solid. 75% yield; MP 232.9-234.1° C.; MS (APCI +ve) m/z=298 (M+H)⁺, 280 (M−H₂O)⁺; IR (KBr disc) ν=3412, 3280, 3207, 3154, 1674, 1618, 1601, 1562, 1539, 1509, 1492, 1412, 1376, 1327, 1244 (N—O), 1171 cm⁻¹; ¹H NMR (D6-DMSO; δDMSO=2.50 ppm) 2.06 (s, 3H, CH₃), 7.13 (bs, 2H, NH₂), 7.63 (d, 2H, J=8.6 Hz, Ar 2′-H and Ar 6′-H), 7.83 (d, 2H, J=8.6 Hz, Ar 3′-H and Ar 5′-H), 7.93 (d, 2H, J=7.3 Hz, Ar 2-H and Ar 6-H), 8.27 (d, 2H, J=7.3 Hz, Ar 3-H and Ar 5-H), 8.37 (s, 1H, N═CH), 10.12 (bs, 1H, NH) ppm

4PYOfb

Yellow crystalline solid. 90% yield; MP 216.1-218.2° C.; MS (APCI +ve) m/z=297 (M+H)⁺, 279 (M−H₂O)⁺; IR (KBr disc) ν=3450, 3324, 3095, 3060, 3029, 2962, 2944, 2898, 2864, 1616, 1527, 1500, 1445, 1400, 1362, 1345, 1330, 1320, 1245 (N—O), 1195 cm⁻¹; ¹H NMR (D6-DMSO; δDMSO=2.50 ppm) 1.31 (s, 9H, C(CH₃)₃), 7.14 (bs, 2H, NH₂), 7.46 (d, 2H, J=8.4 Hz, Ar 3′-H and Ar 5′-H), 7.83 (d, 2H, J=8.4 Hz, Ar 2′-H and Ar 6′-H), 7.94 (d, 2H, J=7.3 Hz, Ar 2-H and Ar 6-H), 8.29 (d, 2H, J=7.3 Hz, Ar 3-H and Ar 5-H), 8.42 (s, 1H, N═CH), 10.12 (bs, 1H, NH) ppm

4PYOfc

Yellow crystalline solid. 84% yield; MP 227.9-229.7° C.; MS (APCI +ve) m/z=377 (M+H)⁺; ¹H NMR (D6-DMSO; δDMSO=2.50 ppm) 3.80 (s, 3H, OCH₃), 5.16 (s, 2H, OCH₂), 7.02 (d, 1H, J=8.4 Hz, Ar 5′-H), 7.13 (bs, 2H, NH₂), 7.30-7.52 (overlapping m, 6H, Ar—H), 7.75 (d, 1H, J=2.2 Hz, Ar 2′-H), 7.93 (d, 2H, J=7.3 Hz, Ar 2-H and Ar 6-H), 8.29 (d, 2H, J=7.3 Hz, Ar 3-H and Ar 5-H), 8.35 (s, 1H, N═CH) ppm

4PYOeh

Yellow crystalline solid. 52% yield; MP 175.8-178.0° C.; MS (APCI +ve) m/z=383/385 (M+H)⁺, 365/367 (M−H₂O)⁺; IR (KBr disc) ν=3371, 3275, 3174, 3092, 1637, 1625, 1617, 1610, 1603, 1560, 1543, 1527, 1509, 1493, 1476, 1458, 1437, 1340, 1285, 1237 (N—O), 1177 cm⁻¹; ¹H NMR (D6-DMSO; δDMSO=2.50 ppm) 7.23 (d, 2H, J=8.5 Hz, ArH), 7.29 (bs, 2H, NH₂), 7.33-7.52 (overlapping m, 5H, ArH), 7.93 (d, 2H, J=7.3 Hz, Ar 2-H and Ar 6-H), 8.27 (d, 2H, J=7.2 Hz, Ar 3-H and Ar 5-H), 8.35 (m, 1H, ArH), 8.80 (s, 1H, N═CH) ppm

4PYOam

Yellow crystalline solid. 58% yield; MP 227.8-228.9° C.; MS (APCI +ve) m/z=340 (M+H)⁺, 323 (M−H₂O)⁺; ¹H NMR (D6-DMSO; δDMSO=2.50 ppm) 7.27 (d, 2H, J=8.5 Hz, ArH), 7.68-7.85 (overlapping m, 4H, Anthryl-H), 8.02 (d, 2H, J=7.2 Hz, Ar 2-H and Ar 6-H), 8.08 (d, 1H, J=7.5 Hz, Anthryl-H), 8.33 (d, 2H, J=7.2 Hz, Ar 3-H and Ar 5-H), 8.59 (s, 1H, Anthryl 10-H), 8.85 (d, 1H, J=7.9 Hz, Anthryl-H), 8.94 (overlapping m, 2H, Anthryl-H), 9.19 (s, 1H, N═CH) ppm

4PYOfd

Yellow crystalline solid. 89% yield; MP 179.3-180.9° C.; MS (APCI +ve) m/z=347 (M+H)⁺, 329 (M−H₂O)⁺; IR (KBr disc) ν=3431, 3275, 3155, 3105, 1614, 1601, 1570, 1490, 1433, 1392, 1260 (N—O), 1181 cm⁻¹; ¹H NMR (D6-DMSO; δDMSO=2.50 ppm) 5.17 (s, 2H, OCH₂), 7.65 (m, 1H, Ar—H), 7.23 (bs, 2H, NH₂), 7.31-7.51 (overlapping m, 7H, Ar—H), 7.67 (m, 1H, Ar—H), 7.94 (d, 2H, J=7.3 Hz, Pyridyl 2-H and Pyridyl 6-H), 8.29 (d, 2H, J=7.3 Hz, Pyridyl 3-H and Pyridyl 5-H), 8.42 (s, 1H, N═CH) ppm

4PYOfe

Yellow crystalline solid. 51% yield; MP 212.9-215.4° C.; MS (APCI +ve) m/z=313 (M+H)⁺; IR (KBr disc) ν=3459, 3273, 3107, 2955, 1628, 1592, 1532, 1485, 1396, 1244 (N—O) cm⁻¹; ¹H NMR (D6-DMSO; δDMSO=2.50 ppm) 1.28 (s, 9H, C(CH₃)₃), 6.86 (d, 1H, J=8.6 Hz, Ar 3′-H), 7.13 (bs, 2H, NH₂), 7.33 (dd, 1H, J=8.6, 2.5 Hz, Ar 4′-H), 7.63 (d, 1H, J=2.5 Hz, Ar 6′-H), 7.94 (d, 2H, J=7.3 Hz, Ar 2-H and Ar 6-H), 8.29 (d, 2H, J=7.2 Hz, Ar 3-H and Ar 5-H), 8.67 (s, 1H, N═CH), 10.60 (bs, 1H, OH) ppm

4PYOff

Yellow crystalline solid. 58% yield; MP 213.8-215.8° C.; MS (APCI +ve) m/z=353 (M+H)⁺, 335 (M−H₂O)⁺; IR (KBr disc) ν=3450, 3282, 2957, 1617, 1533, 1498, 1441, 1395, 1359, 1346, 1253 (N—O) cm⁻¹; ¹H NMR (D6-DMSO; δDMSO=2.50 ppm) 1.33 (s, 18H, 2×C(CH₃)₃), 7.13 (bs, 2H, NH₂), 7.45 (t, 1H, J=1.7 Hz, Ar 4′-H), 7.72 (d, 2H, J=1.8 Hz, Ar 2′ and 6′-H), 7.95 (d, 2H, J=7.1 Hz, Ar 2-H and Ar 6-H), 8.30 (d, 2H, J=7.1 Hz, Ar 3-H and Ar 5-H), 8.46 (s, 1H, N═CH) ppm

4PYOfg

Yellow crystalline solid. 51% yield; MP 195.0-197.2° C.; MS (APCI +ve) m/z=313 (M+H)⁺; IR (KBr disc) ν=3549, 3458, 3307, 3190, 3087, 3064, 2970, 1642, 1600, 1531, 1495, 1426, 1393, 1233 (N—O), 1184 cm⁻¹; ¹H NMR (D6-DMSO; δDMSO=2.50 ppm) 1.43 (s, 9H, C(CH₃)₃), 6.87 (t, 1H, J=7.7 Hz, Ar—H), 7.17 (bs, 2H, NH₂), 7.27-7.35 (overlapping m, 2H, 2×Ar—H), 7.95 (d, 2H, J=7.0 Hz, Ar 2-H and Ar 6-H), 8.30 (d, 2H, J=7.0 Hz, Ar 3-H and Ar 5-H), 8.65 (s, 1H, N═CH), 11.77 (s, 1H, OH) ppm

4PYOfh

Yellow crystalline solid. 42% yield; MP 220.4-223.1° C.; MS (APCI +ve) m/z=369 (M+H)⁺; IR (KBr disc) ν=3624, 3608, 3445, 3260, 2950, 1618, 1486, 1439, 1423, 1227 (N—O), 1171 cm⁻¹; ¹H NMR (D6-DMSO; δDMSO=2.50 ppm) 1.42 (s, 18H, 2×C(CH₃)₃), 6.94 (bs, 2H, NH₂), 7.34 (bs, 1H, OH), 7.62 (s, 2H, Ar 2′ and 6′-H), 7.92 (d, 2H, J=7.Hz, Ar 2-H and Ar 6-H), 8.28 (d, 2H, J=7.1 Hz, Ar 3-H and Ar 5-H), 8.37 (s, 1H, N═CH) ppm

4PYOfi

The product was obtained as a mixture of E/Z isomers (2.4/1 ratio) about the N═C— furyl double bond.

Orange solid. Yield 79%; MS (APCI +ve) m/z=276 (M+H)⁺, 258 (M−H₂O)⁺; IR (KBr disc) ν=3432, 1625, 1509, 1466, 1321, 1247 (N—O), 1172 cm⁻¹; ¹H NMR (D6-DMSO; δDMSO=2.50 ppm) Major product E isomer: 7.39 (d, 1H, J=3.9 Hz, furyl H-3), 7.51 (bs, 2H, NH₂), 7.82 (d, 1H, J=3.9 Hz, furyl H-4), 7.97 (d, 2H, J=7.1 Hz, Ar 2-H and Ar 6-H), 8.31 (d, 2H, J=7.0 Hz, Ar 3-H and Ar 5-H), 8.36 (s, 1H, N═CH) ppm; Minor product Z isomer: 7.59 (d, 1H, J=3.9 Hz, furyl H-3), 7.62 (bs, 2H, NH₂), 7.77 (d, 1H, J=3.8 Hz, furyl H-4), 7.86 (s, 1H, N═CH), 8.00 (d, 2H, J=7.3 Hz, Ar 2-H and Ar 6-H), 8.33 (d, 2H, J=7.0 Hz, Ar 3-H and Ar 5-H) ppm

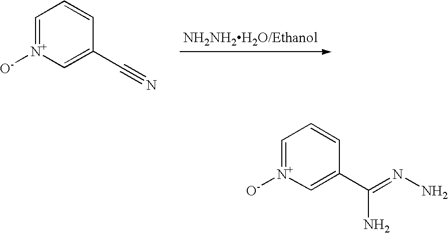

Nicotinonitrile-1-oxide (9.72 g, 81 mmol) was suspended in ethanol (30 mL) and treated with hydrazine monohydrate (20 mL) and stirred at ambient temperature for seven days. The solid material was collected by filtration, washed with ethanol (3×10 mL) and dried under vacuum to give the product as a white powder. Yield 5.774 g, 37.9 mmol, 47%; MP 135.5-138.6° C. (decomposes); MS (APCI +ve) m/z=153 (M+H)⁺, 136 (M−O)⁺IR (KBr disc) vν=3408, 3284, 3174, 1666, 1589, 1564, 1489, 1428, 1390, 1309, 1231 (N—O), 1171, 1121 cm⁻¹; ¹H NMR (D6-DMSO; δDMSO=2.50 ppm) 5.31 (bs, 2H, NH₂), 5.82 (bs, 2H, NH₂), 7.40 (dd, 1H, J=8.1, 6.4 Hz, Ar 5-H), 7.64 (ddd, 1H, J=8.1, 1.0, 0.9 Hz, Ar 4-H), 8.14 (ddd, 1H, J=6.3, 1.0, 0.9 Hz, Ar 6-H), 8.43 (m, 1H, Ar 2-H) ppm.

3PYOab

Yellow powder. Yield 84%; MP 230.1-232.4° C.; MS (APCI +ve) m/z=255 (M+H)⁺, 237 (M−OH)⁺; IR (KBr disc) v=3443, 3292, 3210, 3176, 3137, 3061, 3048, 2913, 1628, 1598, 1570, 1491, 1428, 1409, 1336, 1310, 1249 (N—O), 1177 cm⁻¹; ¹H NMR (D6-DMSO; δDMSO=2.50 ppm) 2.35 (s, 3H, CH₃), 7.20 (bs, 2H, NH₂), 7.25 (d, 2H, J=8.2 Hz, Ar 3′-H and Ar 5′-H), 7.50 (dd, 1H, J=7.9, 6.4 Hz, Ar 5-H), 7.81 (d, 2H, J=7.9 Hz, Ar 2′-H and Ar 6′-H), 7.84 (m, 1H, Ar 4-H), 8.31 (m, 1H, Ar 6-H), 8.42 (s, 1H, N═CH), 8.67 (m, 1H, Ar 2-H) ppm.

3PYOac

Yellow crystalline solid. Yield 70%; MP 213.3-215.5° C.; MS (APCI +ve) m/z=269 (M+H)⁺, 251 (M−OH)⁺; IR (KBr disc) v=3405, 3266, 3128, 3053, 2960, 2928, 2868, 1628, 1599, 1563, 1534, 1490, 1433, 1337, 1309, 1240 (N—O), 1227, 1178 cm⁻¹; ¹H NMR (D6-DMSO; δDMSO=2.50 ppm) 1.02 (t, 3H, J=7.6 Hz, CH₃), 2.65 (q, 2H, J=7.6 Hz, CH₂), 7.20 (bs, 2H, NH₂), 7.28 (d, 2H, J=7.9 Hz, Ar 3′-H and Ar 5′-H), 7.50 (dd, 1H, J=7.9, 6.7 Hz, Ar 5-H), 7.83 (d, 2H, J=8.2 Hz, Ar 2′-H and Ar 6′-H), 7.84 (m, 1H, Ar 4-H), 8.31 (m, 1H, Ar 6-H), 8.42 (s, 1H, N═CH), 8.68 (m, 1H, Ar 2-H) ppm.

3PYOad

Yellow crystalline solid. Yield 78%; MP 214.7-216.8° C.; MS (APCI +ve) m/z=283 (M+H)⁺, 265 (M−OH)⁺; IR (KBr disc) v=3413, 3267, 3205, 3170, 3136, 3059, 3023, 2956, 2866, 1627, 1603, 1565, 1536, 1490, 1431, 1336, 1305, 1240 (N—O), 1225, 1167 cm⁻¹; ¹H NMR (D6-DMSO; δDMSO=2.50 ppm) 1.23 (d, 6H, J=6.7 Hz, CMe₂), 2.93 (septet, 1H CHMe₂), 7.19 (bs, 2H, NH₂), 7.31 (d, 2H, J=8.2 Hz, Ar 3′-H and Ar 5′-H), 7.50 (dd, 1H, J=8.1, 6.5 Hz, Ar 5-H), 7.83 (d, 2H, J=8.2 Hz, Ar 2′-H and Ar 6′-H), 7.84 (m, 1H, Ar 4-H), 8.31 (m, 1H, Ar 6-H), 8.42 (s, 1H, N═CH), 8.68 (m, 1H, Ar 2-H) ppm.

3PYOcq

Yellow crystalline solid. Yield 80%; MP 247.2-248.6° C.; MS (APCI +ve) m/z=369 (M+H)⁺, 351 (M−OH)⁺; IR (KBr disc) v=3477, 3304, 3150, 3088, 2952, 2909, 2867, 1629, 1584, 1542, 1490, 1465, 1438, 1389, 1361, 1307, 1251 (N—O), 1230, 1202, 1173, 1131 cm⁻¹; ¹H NMR (D6-DMSO; δDMSO=2.50 ppm) 1.27 (s, 9H, CMe₃), 1.43 (s, 9H, CMe₃), 7.25 (bs, 2H, NH₂), 7.30 (d, 1H, J=2.4 Hz Ar 4′-H), 7.34 (d, 1H, J=2.3 Hz, Ar 6′-H), 7.54 (dd, 1H, J=8.1, 6.5 Hz, Ar 5-H), 7.84 (ddd, 1H, J=8.1, 1.5, 1.0 Hz, Ar 4-H), 8.33 (ddd, 1H, J=6.4, 1.8, 0.9 Hz, Ar 6-H), 8.66 (s, 1H, N═CH), 8.67 (m, 1H, Ar 2-H), 11.52 (s, 1H, OH) ppm.

3PYOeh

Yellow crystalline solid. Yield 57%; MP 217.6-220.7° C.; MS (APCI +ve) m/z=383 (M+H)⁺, 365 (M−OH)⁺; IR (KBr disc) v=3432, 3306, 3173, 3059, 1630, 1594, 1555, 1531, 1493, 1474, 1435, 1407, 1335, 1248 (N—O), 1229 cm⁻¹; ¹H NMR (D6-DMSO; δDMSO=2.50 ppm) 7.24 (d, 2H, J=8.5 Hz, Ar 3″-H and Ar 5″-H), 7.32-7.52 (overlapping m, 7H, Ar 5-H, Ar 2″-H, Ar 6″-H, 2×Ar′ H, NH₂), 7.83 (m, 1H, Ar 4-H), 8.28-8.38 (overlapping m, 2H, Ar 6-H and Ar′ H), 8.66 (m, 1H, Ar 2-H), 8.79 (s, 1H, N═CH) ppm.

3PYOfb

Recrystallised from ethanol. Yellow crystalline solid. Yield 61%; MP 230.9-233.8° C.; MS (APCI +ve) m/z=297 (M+H)⁺, 279 (M−OH)⁺; IR (KBr disc) v=3412, 3265, 3136, 3061, 2958, 2902, 2865, 1629, 1603, 1560, 1538, 1488, 1430, 1408, 1340, 1238 (N—O), 1225, 1166, 1111 cm⁻¹; ¹H NMR (D6-DMSO; δDMSO=2.50 ppm) 1.30 (s, 9H, CMe₃), 7.23 (bs, 2H, NH), 7.46 (d, 2H, J=8.4 Hz, Ar 3′-H and Ar 5′H), 7.51 (m, 1H, Ar 5-H), 7.81-7.87 (overlapping m, 3H, Ar 4-H, Ar 2′-H and Ar 6′-H), 8.32 (m, 1H, Ar 6-H), 8.42 (s, 1H, N═CH), 8.68 (t, 1H, J=1.4 Hz, Ar 2-H) ppm.

3PYOfc

White powder. Yield 61%; MP 211.2-213.8° C.; MS (APCI +ve) m/z=377 (M+H)⁺, 358 (M−H₂O)⁺; IR (KBr disc) v=3441, 3237, 3106, 3072, 1618, 1596, 1546, 1508, 1486, 1428, 1385, 1351, 1323, 1262 (N—O), 1239, 1165, 1135 cm⁻¹; ¹H NMR (D6-DMSO; δDMSO=2.50 ppm) 3.82 (s, 3H, OMe), 5.17 (s, 2H, OCH₂), 7.03 (d, 1H, J=8.2 Hz, Ar 5′-H), 7.19 (bs, 2H, NH₂), 7.29-7.54 (overlapping m, 7H, Ar 5-H, Ar 6′-H and 5×Ar″ H), 7.81 (d, 1H, J=1.8 Hz, Ar 2′-H), 7.85 (m, 1H, Ar 4-H), 8.32 (ddd, 1H, J=6.4, 1.8, 0.9 Hz, Ar 6-H), 8.36 (s, 1H, N═CH), 8.68 (m, 1H, Ar 2-H) ppm

3PYOfe

Yellow crystalline solid. Yield 52%. MP 206.8-208.4° C.; MS (APCI +ve) m/z=313 (M+H)⁺, 295 (M−OH)⁺; IR (KBr disc) v=3413, 3308, 3122, 2961, 2864, 1652, 1624, 1604, 1585, 1564, 1547, 1422, 1401, 1361, 1341, 1302, 1283, 1264, 1239 (N—O), 1189, 1123 cm⁻¹; ¹H NMR (D6-DMSO; δDMSO=2.50 ppm) 1.27 (s, 9H, CMe₃), 6.85 (d, 1H, J=8.5 Hz, Ar 3′-H), 7.24 (bs, 2H, NH₂), 7.33 (dd, 1H, J=8.5, 2.4 Hz, Ar 4′-H), 7.51 (dd, 1H, J=8.0, 6.8 Hz, Ar 5-H), 7.66 (d, 1H, J=2.4 Hz, Ar 6′-H), 7.84 (m, 1H, 4-H), 8.33 (ddd, 1H, J=6.4, 1.8, 0.9 Hz, Ar 6-H), 8.67 (s, 1H, N═CH), 8.67 (m, 1H, Ar 2-H), 10.55 (bs, 1H, OH) ppm.

3PYOfg

Yellow crystalline solid. Yield 79%. Two polymorphic crystalline forms present: needles MP 228.0-228.9° C., cubes MP 239.4-241.5° C.; MS (APCI +ve) m/z=313 (M+H)⁺, 295 (M−OH)⁺; IR (KBr disc) v=3464, 3285, 3129, 2972, 2942, 2855, 1637, 1604, 1562, 1547, 1480, 1426, 1396, 1342, 1302, 1241 (N—O), 1196, 1162, 1115 cm⁻¹; ¹H NMR (D6-DMSO; δDMSO=2.50 ppm) 1.42 (s, 9H, CMe₃), 6.87 (t, 1H, J=7.63 Hz, Ar′ 5′-H), 7.29 (dd, 1H, J=7.6, 1.5 Hz, Ar 4′-H), 7.29 (bs, 2H, NH₂), 7.34 (dd, 1H, J=7.6, 1.5 Hz, Ar 6′-H), 7.52 (dd, 1H, J=7.9, 6.4 Hz, Ar 5-H), 7.84 (ddd, 1H, J=7.9, 1.5, 0.9 Hz, Ar 4-H), 8.33 (ddd, 1H, J=6.4, 1.8, 0.9 Hz, Ar 6-H), 8.65 (s, 1H, N═CH), 8.68 (m, 1H, Ar 2-H), 11.71 (s, 1H, OH) ppm.

3PYOfj

Recrystallised from ethanol. Yellow crystalline solid. 57% yield; MP 198.5-201.4° C.; MS (APCI +ve) m/z=377 (M+H)⁺, 359 (M−OH)⁺; IR (KBr disc) v=3462, 3356, 3083, 3032, 1610, 1579, 1537, 1509, 1462, 1453, 1417, 1382, 1262 (N—O), 1230, 1163, 1139 cm⁻¹; ¹H NMR (D6-DMSO; δDMSO=2.50 ppm) 3.86 (s, 3H, OMe); 5.15 (s, 2H, OCH₂), 7.09 (d, 1H, J=8.2 Hz, Ar 5′-H), 7.24 (bs, 2H, NH₂), 7.29 (dd, 1H, J=8.2, 1.5 Hz, Ar 6′-H), 7.33-7.49 (overlapping m, 5H, 5×Ar″ H), 7.50 (dd, 1H, J=7.9, 6.4 Hz, Ar 5-H), 7.68 (d, 1H, J=1.5 Hz, Ar 2′-H), 7.85 (m, 1H, Ar 4-H), 8.32 (m, 1H, Ar 6-H), 8.36 (s, 1H, N═CH), 8.68 (m, 1H, Ar 2-H) ppm.

### EXAMPLE 3

### Further Biological Assays

### Clostridium difficile

Compounds 4PYcq and 4PYOfi were screened against one NCTC strain and thirty-two clinical isolates of Clostridium difficile as described below.

Determination of Minimum Inhibitory Concentrations (MIC) and Minimum Bactericidal Concentrations (MBC) Against C. difficile

The antimicrobial compounds were prepared by adding 5.12 mg of each compound to 1 ml of DMSO. From each solution 100 μl was then aseptically transferred into 1 ml of sterile distilled water to give a concentration of 512 μg/ml. 100 μl of each diluted compound was then added to the first well of a sterile 96-well microliter plate. Serial dilutions were performed to give a concentration range spanning from 0 to 512 μg/ml of each compound. The microtiter plate containing the antimicrobials was left to equilibrate in an anaerobic cabinet for 3 hours before the addition of C. difficile cells. An overnight culture of C. difficile (NCTC 11204) was standardized to an OD⁶⁰⁰ of 0.04 (approximately 10⁶ CFU/ml), using Wilkins Chalgren broth and was vortexed for 60 seconds to ensure there was a uniform suspension. 100 μl of standardized culture was then added to each well of the microtiter plate, containing the equilibrated antimicrobials. The final concentrations of the antimicrobials in the wells therefore ranged from 0 to 215 pg/ml and the concentration of the culture was 10⁵ CFU/ml in each well. The microtiter plate was incubated at 37° C., for 48 hours under anaerobic conditions. The MIC was determined as the lowest concentration of antimicrobial agent inhibiting the total growth of C. difficile cells. After incubation, the total solution from each well was aseptically spread onto a separate Wilkins chalgren agar plate. Plates were incubated for 48 hours, at 37° C., under anaerobic conditions. The number of colonies on each plate was counted after incubation and the MBC was determined as the lowest concentration of the antimicrobial agent at which 99.9% of organisms in the original inoculum were killed. The results are shown in Table 10 below.

TABLE 10

| | | |
|---|---|---|
| Strain of | 4PYOfi | 4PYcq |

| Clostridium | MIC | MBC | MIC | MBC |
|---|---|---|---|---|
| difficile | (μg/ml) | (μg/ml) | (μg/ml) | (μg/ml) |
| NCTC 11204 | 2-4 | 2-4 | 4-8 | 4-8 |
| Isolate 1 | 16-32 | 32-64 | 4-8 | 4-8 |
| Isolate 2 | 4-8 | 16-32 | 2-4 | 4-8 |
| Isolate 3 | 8-16 | 8-16 | 2-4 | 2-4 |
| Isolate 4 | 64-128 | 64-128 | 2-4 | 2-4 |
| Isolate 5 | 2-4 | 2-4 | 2-4 | 2-4 |
| Isolate 6 | 16-32 | 16-32 | 2-4 | 2-4 |
| Isolate 7 | 32-64 | 32-64 | 4-8 | 4-8 |
| Isolate 8 | 2-4 | 8-16 | 4-8 | 4-8 |
| Isolate 9 | 128-256 | 128-256 | 4-8 | 4-8 |
| Isolate 10 | 32-64 | 32-64 | 2-4 | 4-8 |
| Isolate 11 | 2-4 | 4-8 | 2-4 | 2-4 |
| Isolate 12 | 2-4 | 4-8 | 4-8 | 4-8 |
| Isolate 13 | 64-128 | 128-256 | 4-8 | 4-8 |
| Isolate 14 | 2-4 | 4-8 | 4-8 | 4-8 |
| Isolate 15 | 2-4 | 8-16 | 4-8 | 4-8 |
| Isolate 16 | 2-4 | 4-8 | 4-8 | 4-8 |
| Isolate 17 | 1-2 | 2-4 | 2-4 | 4-8 |
| Isolate 18 | 1-2 | 1-2 | 2-4 | 2-4 |
| Isolate 19 | 8-16 | 16-32 | 4-8 | 4-8 |
| Isolate 20 | 2-4 | 4-8 | 2-4 | 2-4 |
| Isolate 21 | 16-32 | 16-32 | 4-8 | 4-8 |
| Isolate 22 | 4-8 | 4-8 | 2-4 | 2-4 |
| Isolate 23 | 2-4 | 2-4 | 2-4 | 2-4 |
| Isolate 24 | 4-8 | 4-8 | 4-8 | 4-8 |
| Isolate 25 | 2-4 | 8-16 | 4-8 | 4-8 |
| Isolate 26 | 2-4 | 2-4 | 4-8 | 8-16 |
| Isolate 27 | 16-32 | 16-32 | 4-8 | 4-8 |
| Isolate 28 | 16-32 | 16-32 | 4-8 | 4-8 |
| Isolate 29 | 4-8 | 8-16 | 4-8 | 4-8 |
| Isolate 30 | 16-32 | 16-32 | 2-4 | 2-4 |
| Isolate 31 | 4-8 | 8-16 | 4-8 | 4-8 |
| Isolate 32 | 4-8 | 8-16 | 4-8 | 8-16 |

Propionibacterium acnes

Compounds 4PYcq and 4PYOfi were screened against one NCTC stain and seven clinical isolates of Propionibacterium acnes as described below.

Bacteria Cultures

Bacterial strains of Propionibacterium acnes, stored on beads at −20° C., were plated onto Brain heart infusion (BHI) agar plates. The cultures were incubated anaerobically for 4 days at 37° C.

Minimal Inhibitory Concentration (MIC)

Bacterial suspensions were prepared by diluting bacterial suspensions with BHI to obtain bacterial concentrations approximately 2×10⁶ cfu/ml.

The test antimicrobial compounds were prepared by diluting the compound with appropriate nutrient broth to obtain correct stock solution. 50 μl of BHI were aliquoted onto the wells of the round-bottomed microtitre plate. 50 μl of the antimicrobial compounds were added onto the wells in the first column of the plate and several serial double dilutions were performed along the wells on each row. Following the series of double dilutions of the test compound the bacterial suspension were added onto each well except on the last column which served as a negative control. The lowest concentration of the test compound which inhibited bacterial growth was regarded as MIC of the compound. The results are shown in Table 11 below.

TABLE 11

| P. acnes | 4PYOfi | 4PYcq |
|---|---|---|
| strain | MIC (μg/ml) | MIC (μg/ml) |
| Isolate 1 | 8-16 | 4-8 |
| Isolate 2 | 8-16 | 32-64 |
| Isolate 3 | 8-16 | 2-4 |
| Isolate 4 | 8-16 | 128-256 |
| Isolate 5 | 16-32 | 64-128 |
| Isolate 6 | 8-16 | 64-128 |
| Isolate 7 | 8-16 | >256 |
| NCTC 737 | 16-32 | 64-128 |

Acinetobacter spp.

The compounds 4PYOfi and 4PYcq were shown to be inactive against Acinetobater spp., a Gram-negative bacterium.

1. A method of inhibiting growth of Gram-positive bacteria comprising contacting Gram-positive bacteria in vitro with a compound of formula (I), or a salt or solvate thereof: 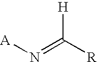
where A is selected from: 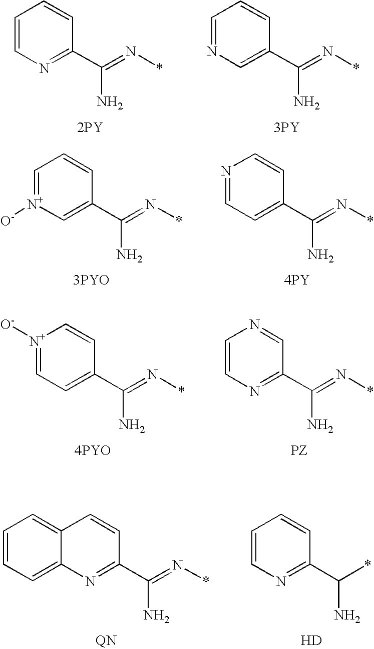
and
R is selected from optionally substituted C₅₋₂₀ aryl, with the proviso that when A is 2PY, then R is not 1,3-dimethylphenyl.

2. The method according to claim 1, wherein the gram-positive bacteria is selected from the classes including Staphylococci, Enterococci, Clostridia, Propionibacteria and Streptococci.

3. The method according to claim 1, wherein the bacteria is resistant to an anti-bacterial agent.

4. The method according to claim 1, wherein the compound is used as a surface disinfectant.

5. The method according to claim 1, wherein R is selected from optionally substituted C₅₋₂₀ carboaryl.

6. The method according to claim 5, wherein R is selected from substituted phenyl, substituted 1-napthyl and substituted or unsubstituted 9-anthryl.

7. The method according to claim 6, wherein R is substituted phenyl.

8. The method according to claim 1, wherein R is selected from optionally substituted C₅₋₂₀ heteroaryl.

9. The method according to claim 8, wherein R is selected from pyrrolyl, imidazolyl, pyridinyl, furanyl, thiophenyl, quinolinyl, 1,4-benzopyronyl, pyrazolyl, isoxazolyl, oxazolyl, thiazolyl, pyridazinyl, pyrimidinyl, pyrazinyl, indolyl, isoindolyl, indazolyl, indolizidinyl, isoquinolinyl and quinazolinyl.

10. The method according to claim 1, wherein R is substituted by one or more substituents selected from hydroxyl, C₁₋₆ straight, branched or cyclic alkyl, C₁₋₆ straight, branched or cyclic alkoxy or alkylthio, C₁₋₆ straight, branched or cyclic alkylcarbonyloxy, C₁₋₆ straight, branched or cyclic alkoxycarbonyl, cyano, amino, nitro, halo, phenyl, benzyl, and phenyl(C₁₋₆)alkyloxy, in each case the phenyl moiety itself being substituted or unsubstituted.

11. The method according to claim 10, wherein R is substituted by one or more substituents selected from hydroxyl, methoxy, ^{t}butyl, 1,1-dimethylpropyl, substituted or unsubstituted phenylthio, aminoalkyloxy, iodo, bromo and nitro.

12. The method according to claim 1, wherein R is at least di-substituted.

13. The method according to claim 12, wherein at least one of said substituents is hydroxyl or ^{t}butyl.

14. The method according to claim 1, wherein A is selected from 2PY, 4PY and HD.

15. A method treating a patient afflicted with a Gram-positive bacterial infection comprising administering to the patient an effective amount of a compound according to formula (I), or a salt or solvate thereof 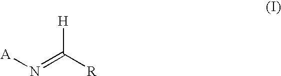
where A is selected from: 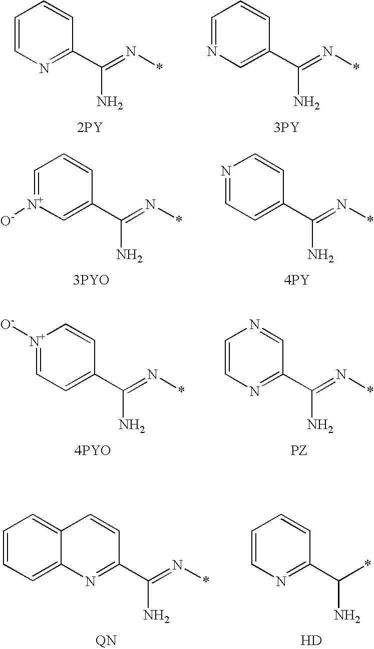
and
R is selected from optionally substituted C₅₋₂₀ aryl, with the proviso that when A is 2PY, then R is not 1,3-dimethylphenyl.

16. The method according to claim 15, wherein the compound is administered topically.

17. A compound of formula (I), or a salt or solvate thereof: 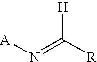
where A is selected from: 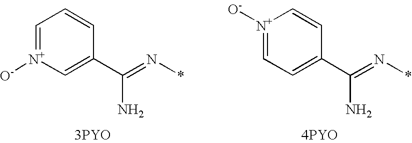
and
R is selected from optionally substituted C₅₋₂₀ aryl.

18. A compound according to claim 17, wherein R is selected from optionally substituted C₅₋₂₀ carboaryl.

19. A compound according to claim 17, wherein R is optionally substituted phenyl.

20. A compound according to claim 19, wherein R is 2-hydroxy, 3,5-di^{t}butyl-phenyl.

21. A method of treating patient afflicted with a Gram-positive bacterial infection comprising administering to the patient an effective amount of a compound according to claim 17, or a salt or solvate thereof.

22. A compound 4PYcq, or a salt or solvate thereof:

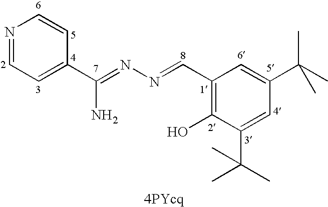

23. A method of treating a patient afflicted with a Gram-positive bacterial infection comprising administering to the patient an effective amount of a compound according to claim 22, or a salt or solvate thereof.

24. A compound of formula (I), or salts or solvates thereof: 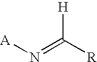
where:
(a) A is selected from: 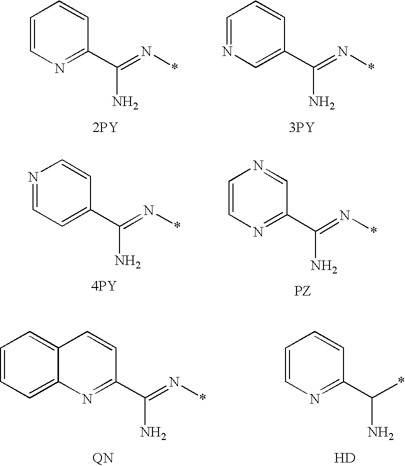
and R is m-NO₂-phenyl, where the phenyl further bears a hydroxyl substituent, and is optionally further substituted;
(b) A is 3PY and R is optionally substituted C₅₋₂₀ carboaryl;
(c) A is 3PY or PY and R is 4-t-pentyl phenyl, where the phenyl is optionally further substituted;
(d) A is 2PY, 3PY, 4PY, PZ, QN or HD and R is trihydroxyphenyl;
(e) A is 2PY, 3PY, 4PY, PZ or QN and R is optionally further substituted dihydroxyphenyl;
(f) A is 2PY, 3PY, 4PY, PZ, QN or HD and R is p-OH phenyl where the phenyl bears a further hydroxy substituent, and is optionally further substituted;
(g) A is 2PY, 3PY, 4PY, PZ, QN or HD and R is optionally substituted anthracenyl;
(h) A is 2PY, 3PY, 4PY, PZ, QN or HD and R is 3-,5-di-t-butyl phenyl, where the phenyl further bears a hydroxy substituent;
(i) A is 4PY and R is thioether phenyl; or
(j) A is HD and R is napthyl.

25. A method of treating a patient afflicted with a Gram-positive bacterial infection comprising administering to the patient an effective amount of a compound according to claim 24, or a salt or solvate thereof.
